(19) United States
(12) Patent Application Publication
(10) Pub. No.: US 2020/0255818 A1
(43) Pub. Date: Aug. 13, 2020

(54) POLYMERIC ENCAPSULATION OF WHOLE CELLS AS BIOREACTORS

(71) Applicant: Lawrence Livermore National Security, LLC, Livermore, CA (US)

(72) Inventors: Jennifer M. Knipe, Oakland, CA (US); Sarah Baker, Dublin, CA (US); Joshua R. Deotte, Livermore, CA (US); Fang Qian, Santa Cruz, CA (US)

(21) Appl. No.: 16/862,342

(22) Filed: Apr. 29, 2020

Related U.S. Application Data
(63) Continuation-in-part of application No. PCT/US2018/058214, filed on Oct. 30, 2018.
(60) Provisional application No. 62/579,067, filed on Oct. 30, 2017.

(51) Int. Cl. C12N 11/089; C12M 1/00; C12N 11/04

(52) U.S. Cl. CPC C12N 11/089; B33Y 10/00; C12N 11/04; C12M 21/02

(57) ABSTRACT

In one inventive concept, a mixture for forming polymer-encapsulated whole cells includes a pre-polymer, a photoinitiator, and a plurality of whole cells. In another inventive concept, a product includes a structure including a plurality of whole cells encapsulated in a polymer, where the polymer is cross-linked.

### RELATED APPLICATIONS

This application is a Continuation in Part of International Application No. PCT/US18/58214 filed Oct. 30, 2018 and claims priority to U.S. Provisional Patent Application No. 62/579,067 filed Oct. 30, 2017, both of which are herein incorporated by reference.

The United States Government has rights in this invention pursuant to Contract No. DE-AC52-07NA27344 between the United States Department of Energy and Lawrence Livermore National Security, LLC for the operation of Lawrence Livermore National Laboratory.

### FIELD OF THE INVENTION

The present invention relates to bioreactors, and more particularly to biocatalytic microcapsules that include whole cell-embedded multicomponent polymers that may provide improved surface area and mass transport to facilitate conversion of target gases using living native microbes and/or engineered microbes embedded and/or printed in the multicomponent polymers.

### BACKGROUND

Advances in oil and gas extraction techniques have made vast new stores of natural gas (composed primarily of methane) available for use. However, substantial quantities of methane are leaked, vented, or flared during these operations. Indeed, methane emissions contribute about a third of current net global warming potential. Compared to other hydrocarbons, and especially compared to the oil that is co-produced in hydrofracturing operations, methane has a much lower market value due to difficulty in methane storage and transport, and because methane has limited use as a transportation fuel.

Conversion of methane to methanol via conventional industrial technologies, such as steam reformation followed by the Fischer-Tropsch process, operate at high temperature and pressure, depend on a large number of unit operations, and yield a range of products. Consequently, conventional industrial technologies have a low efficiency of methane conversion to final products and can only operate economically at very large scales. A technology to efficiently convert methane to other hydrocarbons is highly sought after as a potentially profitably way to convert “stranded” sources of methane and natural gas (e.g., sources that are small, temporary, not close to a pipeline, etc.) to liquids for further processes.

Most chemical reactions of interest for clean energy are routinely carried out in nature. These reactions include the conversion of sunlight to chemical energy, the transfer of carbon dioxide into and out of solution, the selective oxidation of hydrocarbons (including methane to methanol), the formation of C—C bonds (including methane to ethylene), and the formation and dissolution of Si—O bonds (including enhanced mineral weathering). Conventional industrial approaches to catalyze these reactions are either inefficient or have yet to be developed.

Biological methane conversion relies on significantly lower energy and capital costs than chemical conversion. Certain enzymes have been identified that carry out each of the aforementioned reactions. Unfortunately, industrial biocatalysis is primarily limited to the synthesis of low-volume, high-value products, such as pharmaceuticals, due to narrow operating parameters in order to preserve biocatalyst activity. Thus, enzyme-catalyzed reactions are typically carried out in a fermenter apparatus, in particular a closed tank reactor with continuous stirring (“stirred”) configured to use bubbled gases for mass transfer. FIG. 1, illustrates a conventional stirred-tank reactor 100, which includes a motor 102, an input/feed tube 104, a cooling jacket 106, one or more baffles 108, an agitator 110, one or more gas spargers 112, and an aqueous medium 114. Gas exchange in the stirred-tank reactor 100 is achieved by bubbling from the sparger(s) 112 at the bottom of the aqueous medium 114 and gas collection above said aqueous medium 114.

Using a stirred-tank reactor tends to be restricted by the extra care needed to maintain a narrow set of conditions to favor the desired metabolic pathways rather than competing pathways and competing organisms. Moreover, stirred-tank reactors are energy inefficient by relying on batch processing, suffering loss of catalytic activity by enzyme inactivation, and exhibiting slow rates of throughput due to low catalyst loading and limited mass-transfer.

Immobilizing enzymes on inert, artificial materials may allow reuse of enzymes (e.g., reactivation of the enzymes) in stirred-tank reactors and thus improve stability in reactor conditions. As shown in FIG. 2, one conventional approach is to immobilize enzymes 202 on the surface of an inert material 204. Other conventional approaches may involve immobilizing enzymes on the surface of accessible pores of inert materials. However, such conventional enzyme immobilization techniques also suffer from lower volumetric catalyst densities, low throughput rates, and do not have routes for efficient gas delivery or product removal.

Moreover, the use of enzymes and enzymatic components results in limited mass transfer of gas phase reactants to the biocatalyst, and, unfortunately, depends on expensive cofactors such as the electron donor nicotinamide adenine dinucleotide, (NADH) for specific stoichiometric conversion of methane to methanol.

Accordingly, it would be advantageous to develop a novel system and related techniques for effective conversion of methane and/or other common sources of gaseous carbon-containing materials without the use of expensive cofactors such as NADH. Moreover, it would be desirable to develop a system that uses less expensive cofactors and/or coenzymes to provide a scalable carbon capture application and functionality.

### SUMMARY

In one inventive concept, a mixture for forming polymer-encapsulated whole cells includes a pre-polymer, a photoinitiator, and a plurality of whole cells.

In another inventive concept, a product includes a structure including a plurality of whole cells encapsulated in a polymer, where the polymer is cross-linked.

In yet another inventive concept, a bioreactor includes a three-dimensional structure, where the three-dimensional structure is comprised of a gas-permeable material, and polymer-encapsulated whole cells. In addition, at least one wall of the three-dimensional structure is infilled with polymer-encapsulated whole cells.

Other aspects and implementations of the presently described inventive concepts will become apparent from the following detailed description, which, when taken in conjunction with the drawings, illustrate by way of example the principles of the invention.

### BRIEF DESCRIPTION OF THE DRAWINGS

For a fuller understanding of the nature and advantages of the present invention, reference should be made to the following detailed description read in conjunction with the accompanying drawings.

FIG. 1 is a schematic representation of a conventional stirred-tank reactor, according to the prior art.

FIG. 2 is a schematic representation of enzymes immobilized on an exterior surface of an inert material, according to the prior art.

FIG. 3 is a schematic representation of enzymatic reactive components and/or whole cells embedded within a polymeric network, according to one aspect.

FIG. 4 is a process flow illustrating a method for embedding enzymatic reactive components and/or whole cells within a two phase (AB) polymer network, according to one aspect.

FIG. 5 is a process flow illustrating a method for embedding enzymatic reactive components and/or whole cells within a two phase (AB) polymer network, according to another aspect.

FIG. 6A is schematic representation of a bioreactor comprising a hollow tube network/lattice configured to optimize mass transfer, according to one aspect.

FIG. 6B part (a) is an image of a silicone structure 3D printed using projection microstereolithography, according to one aspect.

FIG. 6B part (b) is an image of a silicone structure 3D printed using direct ink writing, according to one aspect.

FIG. 7A is a flowchart of a method for forming a bioreactor via 3D printing, according to one aspect.

FIG. 7B is a simplified schematic of direct-ink-writing with novel ink formulations comprised of nanocellulose crystals, PEGDA, and yeast, according to one aspect.

FIG. 7C are images of formed PEG-pMMO 3D structures using 3D printing techniques. Part (a) illustrates a structure formed/patterned according to a direct ink write (DIW) process, part (b) illustrates a structure formed/patterned according to a projection microstereolithography (PμSL) process, according to some aspects.

FIG. 8A is a plot of CO₂ (product) to methane (reactant) ratios of UV-cured and uncured polymer formulations with methanotroph cells, according to one aspect.

FIG. 8B is a plot of CO₂ (product) to methane (reactant) ratio of methanotroph cells in various geometries and structures, according to one aspect.

FIG. 8C is a plot of Methane consumption of methanotroph cells at varying cell densities in solution as compared to a lattice structure, according to another aspect.

FIG. 9 is a process flow illustrating a method for forming an acrylate-functionalized polyethylene glycol hydrogel comprising particulate methane monooxygenase (pMMO), according to one aspect.

FIG. 10A is a plot illustrating pMMO retention by weight in a PEGDA hydrogel as a function of the volume percentage of PEGDA present during polymerization, where 150 μg of pMMO is initially included within the PEGDA hydrogel.

FIG. 10B is a plot illustrating pMMO activity in a PEGDA hydrogel as a function of the volume percentage of PEGDA present during polymerization, where 150 μg of pMMO is initially included within the PEGDA hydrogel.

FIG. 10C is a plot illustrating pMMO retention by weight in a PEGDA hydrogel as a function of the amount of pMMO (μg) included during polymerization.

FIG. 10D is a plot illustrating the activity of PEGDA-pMMO and a pMMO control as a function of the amount of pMMO (μg) included during the activity assay.

FIG. 11A is a plot illustrating the activity of the PEGDA-pMMO hydrogel after reusing said hydrogel over multiple cycles.

FIG. 11B is a plot illustrating the amount of methanol (nmoles) produced per mg of pMMO for both as-isolated membrane bound pMMO and PEGDA-pMMO over twenty cycles of methane activity assay.

FIG. 12A is a schematic representation of a continuous flow-through PEGDA-pMMO hydrogel bioreactor, according to one aspect.

FIG. 12B is a plot illustrating the amount of methanol (nmole) produced per mg of pMMO in the PEGDA-pMMO hydrogel bioreactor of FIG. 12A.

FIG. 13 is a plot illustrating the dependence of PEGDA-pMMO activity on surface area to volume ratio for a PEGDA-pMMO hydrogel bioreactor.

FIG. 14A is a schematic diagram showing methanotrophs can convert methane gas to produce a wide range of chemicals.

FIG. 14B is a schematic diagram of a simplified metabolism pathway of succinic acid production.

FIG. 14C is a scanning electron microscope image of methanotroph cells, according to one embodiment.

FIG. 15A are images of acrylate-functionalized PEG hydrogel in vials, according to one embodiment. Part (a) is an image of vials before curing, blank in left vial, and containing cells in right vial. Part (b) is an image of vials after curing, blank in left vial, and containing cells in right vial.

FIG. 15B is an image of hydrogel discs with increasing optical density (OD) of 0, 10, 20, and 40, according to one embodiment.

FIG. 15C are images of fluorescent-dyed cells showing viability of cells following one week, according to one embodiment. Part (a) shows a field of liquid-cultured cells, part (b) shows a field of encapsulated cells in hydrogel.

FIG. 15D are plots of the viability of cells over time. Part (a) is a plot of the comparison of encapsulated cells in hydrogel and cells suspended in liquid (suspension) over 6 days, part (b) is a plot of encapsulated cells in hydrogel over one month.

FIG. 16A is a schematic drawing of the molecular structure of PEGDA.

FIG. 16B is a plot of FT-IR spectra of PEGDA having different molecular weights, according to various approaches.

FIG. 16C is a plot of the viability of cells encapsulated with PEGDA hydrogel at different molecular weights over seven days, according to one embodiment.

FIG. 17A part (a) is a schematic drawing of a conventional stir tank bioreactor for liquid culture, part (b) is a schematic drawing of a magnified view of the liquid culture, and part (c) is a schematic drawing of a further magnified view of gas absorption in the liquid of suspended cells.

FIG. 17B part (a) is a schematic drawing of a hollow fiber membrane reactor based on immobilized live cells, according to one embodiment. Part (b) is a schematic drawing of a magnified view of the gas transfer across the fiber membrane toward the liquid, according to one embodiment.

FIG. 18A is an image of a permeability cell positioned in a water bath, according to one approach.

FIG. 18B is a schematic drawing of the permeability cell configuration, according to one embodiment.

FIG. 18C is a plot of the flux of dissolved carbon dioxide (CO₂) across the hydrogel membrane as a function of membrane thickness, according to one embodiment.

FIG. 19A is a schematic drawing of printing a scaffold using projection microstereolithography (PμSL) technology, according to one embodiment.

FIG. 19B part (a) is a perspective view of a computer-aided design (CAD) drawing of a scaffold, part (b) is a top down view of the CAD drawing of a scaffold, part (c) is a magnified side view of the CAD drawing of a scaffold, part (d) is an image of a perspective view of a printed scaffold, part (e) is an image of a top down view of a printed scaffold, and part (f) is an image of a magnified side view of a printed scaffold, according to one embodiment.

FIG. 19C is a schematic drawing of infilling a printed scaffold with encapsulated cells, according to one embodiment. Part (a) is a drawing of the porous scaffold, part (b) shows the porous scaffold infiltrated with live cells suspended in hydrogel, and part (c) the infiltrated scaffold infiltrated with live cells suspended in hydrogel is cured. Parts (b) and (c) show an inset representing a magnified view of the live cells encapsulated with hydrogel as the infiltrant.

FIG. 19D is a schematic drawing of simulated methane gas diffusion profile across the scaffold wall, according to one embodiment. Part (a) shows a wire frame of an apparatus and part (b) shows the methane concentration profile a vertical cross-section of the sidewall of the hydrogel cylinder of the apparatus of part (a).

FIG. 19E is a plot of methane gas consumption rates over 24 hours as a function of optical density (OD) and geometries, according to various approaches.

FIG. 19F is a comparison of methane gas consumption at OD 20 in different geometries over a month, according to one embodiment.

FIG. 20 is a comparison of succinate production as a function of optical density and geometries, according to various approaches.

### DETAILED DESCRIPTION

The following description is made for the purpose of illustrating the general principles of the present invention and is not meant to limit the inventive concepts claimed herein. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc.

It must also be noted that, as used in the specification and the appended claims, the singular forms “a,” “an” and “the” include plural referents unless otherwise specified.

As also used herein, the term “about” when combined with a value refers to plus and minus 10% of the reference value. For example, a length of about 100 nm refers to a length of 100 nm ±10 nm.

As further used herein, the term “fluid” may refer to a liquid or a gas.

Further, as used herein, all percentage values are to be understood as percentage by weight (wt. %), unless otherwise noted. Moreover, all percentages by weight are to be understood as disclosed in an amount relative to the bulk weight of an organic plastic scintillator material, in various approaches.

Unless expressly defined otherwise herein, each component listed in a particular approach may be present in an effective amount. An effective amount of a component means that enough of the component is present to result in a discernable change in a target characteristic of the ink, printed structure, and/or final product in which the component is present, and preferably results in a change of the characteristic to within a desired range. One skilled in the art, now armed with the teachings herein, would be able to readily determine an effective amount of a particular component without having to resort to undue experimentation.

The following description discloses several preferred structures formed via direct ink writing (DIW), extrusion freeform fabrication, or other equivalent techniques and therefore exhibit unique structural and compositional characteristics conveyed via the precise control allowed by such techniques.

The following description discloses several preferred inventive concepts of polymeric encapsulation of whole cells as bioreactors and/or related systems and methods.

In one general inventive concept, a mixture for forming polymer-encapsulated whole cells includes a pre-polymer, a photoinitiator, and a plurality of whole cells.

In another general inventive concept, a product includes a structure including a plurality of whole cells encapsulated in a polymer, where the polymer is cross-linked.

In yet another general inventive concept, a bioreactor includes a three-dimensional structure, where the three-dimensional structure is comprised of a gas-permeable material, and polymer-encapsulated whole cells. In addition, at least one wall of the three-dimensional structure is infilled with polymer-encapsulated whole cells.

A list of acronyms used in the description is provided below.

3D Three-dimensional

C Celsius

CO₂ Carbon dioxide

Da Daltons

DIW Direct ink writing

FT-IR Fourier transform infrared spectroscopy

kDa kiloDaltons

mL milliliter

mM millimole

MMO Methane monooxygenase

NADH Nicotinamide adenine dinucleotide

NLP Nano-lipo-protein

nm nanometer

nmoles nanomoles

O Oxygen

OD Optical density

PEG Polyethylene glycol

PEGDA Polyethylene glycol diacrylate

PEGTA Polyethylene glycol tetra-acrylate

pMMO Particulate methane monooxygenase

PμSL Projection microstereolithography

Si Silicon

UV Ultraviolet

wt. % weight percent

As discussed previously, enzymes have been identified that catalyze virtually all of the reactions relevant to clean energy, such as selective transformations among carbon fuels, gas to liquids technology, storage of solar energy, exchange of CO₂, formation and dissolution of silicates, and neutralization of wastes. However, a number of factors limit industrial enzyme biocatalysis to low-volume, high-value products (e.g. pharmaceuticals) such as narrow operating parameters to preserve biocatalyst activity, slow rates of throughput due to low catalyst loading, limited mass transfer, and susceptibility to contamination and poisoning.

Accordingly, many biocatalysis processes are currently carried out in single phase, aqueous media using such processes as stirred-tank reactors. However, stirred-tank reactors are energy inefficient, use batch processing, and have poor mass transfer characteristics. While techniques have emerged to improve the stability and allow reuse of enzymes in stirred-tank reactors, such techniques involve immobilizing the enzymes solely on the exterior surface(s) of an inert material or on the exterior surface(s) of the pores of an inert material. Unfortunately, these conventional immobilization techniques still fail to rectify the slow throughput rates and limited mass transfer associated with current biocatalysis processes.

The only biological catalysts isolated to selectively facilitate conversion of methane gas to liquid products under ambient conditions are methane monooxygenase (MMO) enzymes from certain soil microbes. Biological methane conversion uses significantly lower energy and has fewer capital costs than chemical conversion, however, current stirred-tank bioreactors are limited by mass transfer of gas phase reactants to the biocatalysts, buildup of product within the biomass, and/or the need for expensive cofactors to drive the biocatalysis. To overcome these drawbacks, the presently disclosed inventive concepts include development of advanced manufactured bioreactors encapsulating whole cells thereby enabling use of the full cell proteome to tailor product selectivity and to eliminate previously necessary cofactors, while 1) providing control over reactor size and geometry to overcome mass transfer limitations and 2) enabling three-dimensional (3D) printing with formulations that are compatible with preferred additive manufacturing technologies such as projection microstereolithography (PμSL) and direct ink-write (DIW).

Furthermore, encapsulating whole cells within the bioreactor material may enable conversion to products more valuable than the methanol product currently being generated from methane by the biocatalytic material of other approaches described herein. Moreover, encapsulation of whole cells within a printable material may allow improvement of gas-to-liquid mass transfer via control of the geometry and material chemistry, which is a current limitation of growing the cells in a conventional stirred-tank reactor.

To address the problem of limited yields with conventional biological processes, the approaches described herein offer the advantage of decoupling biomass and bioproduct accumulation by encapsulation of whole cells within the material of the bioreactor. Moreover, these approaches may provide modular and scalable bioreactors designed for stranded natural gas upgrading, so that in terms of economy, this otherwise flared or vented gas may be collected as a liquid product suitable for fuels and chemicals.

In some approaches, aspects disclosed herein are directed to a novel class of bioreactor that includes a membrane comprising one or more types of whole cells and or reactive enzymes, enzyme-containing cell fragments embedded within and throughout the depth of a multicomponent polymer network. In various approaches, this multicomponent polymer network may comprise two or more polymer types, or a mixture of a polymer and inorganic material.

Preferably, the membrane includes permeable, multi-component polymers that may serve as a mechanical support for the embedded enzymes and/or whole cells. In addition, the permeable, multi-component polymers of the membrane may serve as functional materials configured to perform one or more additional functions of the bioreactor, such as: efficiently distributing reactants and removing products, exposing the embedded whole cells and/or enzymes to high concentrations of reactants, separating reactants and products, forming high surface area structures for exposing the whole cells and/or embedded enzymes to reactants, supplying electrons in hybrid enzyme-electrochemical reactions, consolidating enzymes and/or whole cells with co-enzymes in nanoscale subdomains for chained reactions, etc. In additional approaches, the membrane described herein may be molded into shapes/features/structures (e.g., hollow fibers, micro-capsules, hollow tube lattices, spiral wound sheets, etc.) to optimize the bioreactor geometry for mass transfer, product removal, and continuous processing.

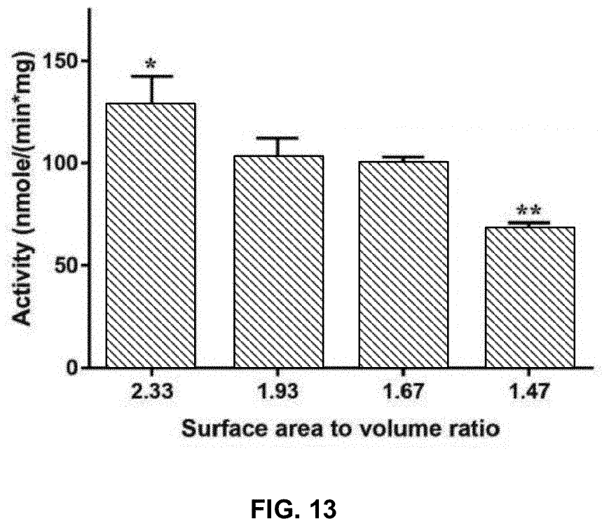

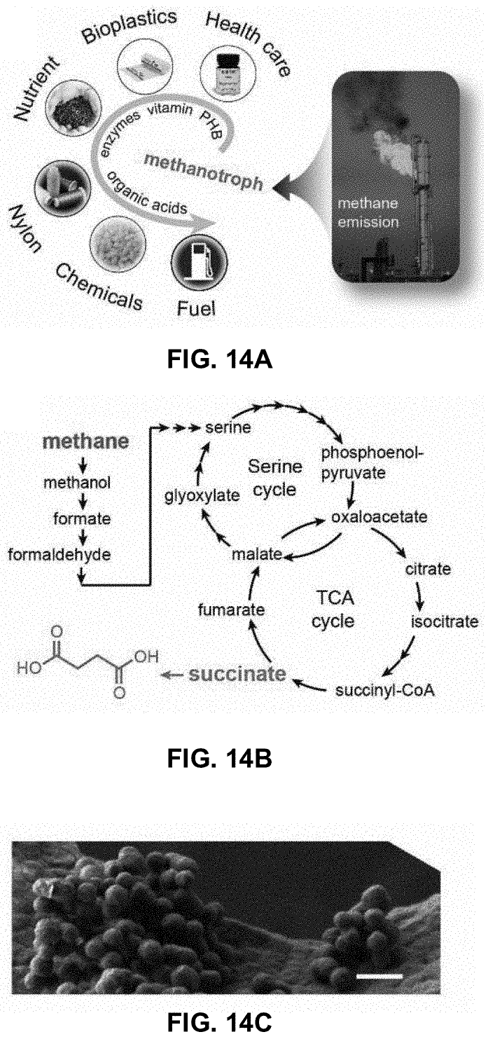

The novel class of bioreactor disclosed herein may be especially suited to catalyze reactions that occur at phase boundaries, e.g., gas to liquid, liquid to gas, polar to non-polar, non-aqueous to aqueous, etc. Table 1 lists products that may be formed in bioreactors as disclosed herein. Accordingly, the novel class of bioreactors disclosed herein may be useful for reactions in clean energy applications that involve a gas-phase reactant or product. FIG. 14A is a schematic drawing that illustrates the products that may be formed in a bioreactor as described herein that includes enzymes, encapsulated (e.g., embedded) whole cells having enzyme capabilities, methanotroph activity, etc. For

TABLE 1
Products Formed in Bioreactors as described herein

| Product | Formula | Application |
|---|---|---|
| Acetic Acid | C₂H₄O₂ | Chemical |
| Adipic Add | C₆H₁₀O₄ | Chemical |
| Formate | CH₂O₂ | Chemical |
| Glycogen | C₆H₁₂O₆ | Chemical |
| Lactic Acid | C₃H₆O₃ | Chemical |
| Muconic Acid | C₆H₆O₄ | Chemical |
| Succinic Acid | C₄H₆O₄ | Chemical |
| Sucrose | C₁₂H₂₂O₁₁ | Chemical |
| Astaxanthin | C₄₀H₅₂O₄ | Healthcare |
| Ectoine | C₆H₁₀N₂O₂ | Healthcare |
| Isoprene | C₅H₈ | Fuel/Rubber |
| Lipid | C₁₅—C₁₈ | Fuel |
| Methanol | CH₄O | Fuel |
| PHB | C₄H₈O₂ | Fuel/Plastic |
| Single Cell Protein | | Nutrient |

example, and not meant to be limiting in any way, methane to methanol conversion, CO₂ absorption, oxidation reactions with O₂, reduction reactions with H₂ or methane, CO₂ conversion to synthetic fuel, etc. In addition, bioreactors may include reactions in the chemical and pharmaceutical industries that involve treatment of non-polar organic compounds with polar reactants (or vice versa).

In one approach, the bioreactor includes engineered whole cells that may convert methane to produce succinate as one of the possible products. As shown in the schematic pathways of FIG. 14B, whole cells of the bioreactor may consume methane to ultimately produce succinate via a serine cycle and TCA cycle.

The following description discloses several general, specific, and preferred aspects relating to bioreactors based on enzyme-embedded and/or whole-cell-embedded multicomponent polymers arranged as nano-, micro- and/or millimeter-structures. For example, in one approach, a bioreactor may include whole-cell-embedded polymers as shown in the image of a scanning electron micrograph (SEM) of FIG. 14C.

In one general aspect, a membrane includes a polymeric network configured to separate a first fluid and a second fluid, where the first and second fluids are different; and a plurality of whole cells embedded within the polymeric network.

In another general aspect, a bioreactor includes a lattice of three-dimensional (3D) structures, each structure including a membrane having a polymeric network configured to separate a first fluid and a second fluid, where the first and second fluids are different. In addition, the membrane includes whole cells embedded within the polymeric network.

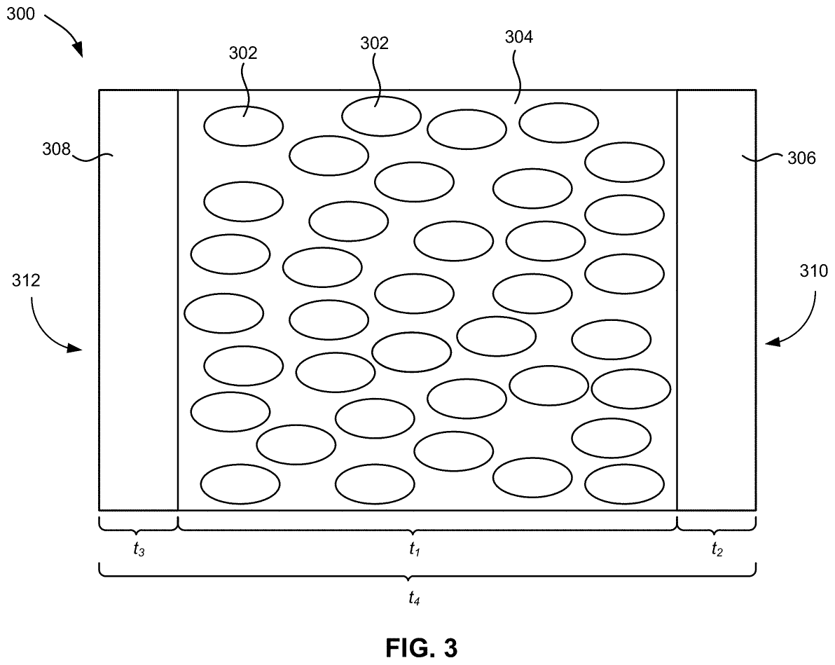

Referring now to FIG. 3, a membrane 300 particularly suitable for use in a bioreactor is shown according to one aspect. As an option, the membrane 300 may be implemented in conjunction with features from any other aspect listed herein, such as those described with reference to the other FIGS. Of course, the membrane 300 and others presented herein may be used in various applications and/or in permutations which may or may not be specifically described in the illustrative aspects listed herein. For instance, the membrane 300 may be used in any desired environment and/or include more or less features, layers, etc. than those specifically described in FIG. 3.

As shown in FIG. 3, the membrane 300 includes a plurality of components 302 embedded within a polymer network 304. In some approaches, the components 302 of the membrane 300 includes a plurality of whole cells. In some approaches, the components 302 of the membrane 300 includes a plurality of enzymatic reactive components. In some approaches, the components 302 of the membrane 300 include whole cells and enzymatic reactive components.

In various approaches, the components 302, whole cells and/or enzymatic reactive components, may comprise about 1% to 80% of the mass of the polymer network 304. The components 302, whole cells and/or enzymatic reactive components, may be configured to catalyze any of the reactions described herein, and in particular reactions that take place at phase boundaries (e.g., gas to liquid, liquid to gas, polar to non-polar, non-aqueous to aqueous, etc.).

In some exemplary approaches, the components 302 are whole living cells. A whole living cell is defined as a cell capable of metabolic activity. In some approaches, a whole living cell may be capable of cell division. In some approaches, a whole living cell is an intact proteome. In various approaches, a whole living cell is a prokaryotic cell. In other approaches, a whole living cell is a eukaryotic cell. In some approaches, the components 302 are bacteria that obtain their carbon and energy from methane. Methanotrophs are gamma proteobacteria that obtain their carbon and energy from methane. In general, any suitable methanotrophic and/or methylotrophic species or other organism known in the art to function as a carbon capture/conversion/consumption agent may be employed. Exemplary organisms include members of the methylococcus and/or methylomicrobium, genus, particularly Methylococcus. capsulatus (M. capsulatus) Bath and Methylomicrobium buryatense (M. buryatense).

M. buryatense is a methanotrophic strain suitable for large-scale production of various chemical and fuels. An engineered strain of M. buryatense may enable conversion of methane to lactate, a precursor to bioplastics, according to various approaches. Immobilizing dried whole M. buryatense in various materials describe herein may remove a need for a reducing agent. In some approaches, incorporating whole cells (e.g., each cell as an entire proteome) may allow electron transfer between coenzymes thereby removing the need for a cofactor such as NADH. Without wishing to be bound by any theory, lactate production may be demonstrated in engineered M. buryatense without the addition an exogenous cofactor to participate in electron transfer.

Engineered strains of M. buryatense have been shown to convert about 75% of carbon into lactate. In related studies as described herein, enzymes in a freeze-dried related organism M. capsulatus proteome have been shown to be highly active. In some approaches, whole cells of M. buryatense may be immobilized in a printable polymeric material while maintaining biocatalytic activity.

Of course, it should be understood that the suitable organisms and applications for the presently disclosed inventive concepts are not limited to carbon capture or carbon metabolism. For instance, in other approaches whole cells may include or be yeast (e.g. species in the saccharomyces genus) and the bioreactors may be utilized in applications for generating, e.g., ethanol.

Encapsulation of Whole Cells

In one aspect, the polymeric network 304 embedded with components 302 may represent polymer-encapsulated whole cells. In one approach, a mixture for forming polymer-encapsulated whole cells may include a pre-polymer, a photoinitiator, and a plurality of whole cells. In one approach, immobilization of whole cells may include whole M. capsulatus Bath and M. buryatense cells encapsulated in various polymers and/or biomaterials.

In some exemplary approaches, the whole cells are whole living cells. In some approaches, the whole cells are bacteria that obtain their carbon and energy from methane. In some approaches, the whole cells may have a characteristic to convert a chemical reactant to product. For example, the chemical reactant is a gas and the whole cells convert the gas to a product, where the product is a liquid. In some approaches, the whole cells are configured to convert methane to methanol. In some approaches, exemplary organisms may be methanotrophic organisms and methylotrophic organisms and include members of the methylococcus and/or methylomicrobium, genus, particularly M. capsulatus Bath and M. buryatense.

In other approaches, the whole cells may be freeze-dried living whole cells. For example, whole cells may include or be yeast (e.g. species in the saccharomyces genus) and the bioreactors may be utilized in applications for generating, e.g., ethanol.

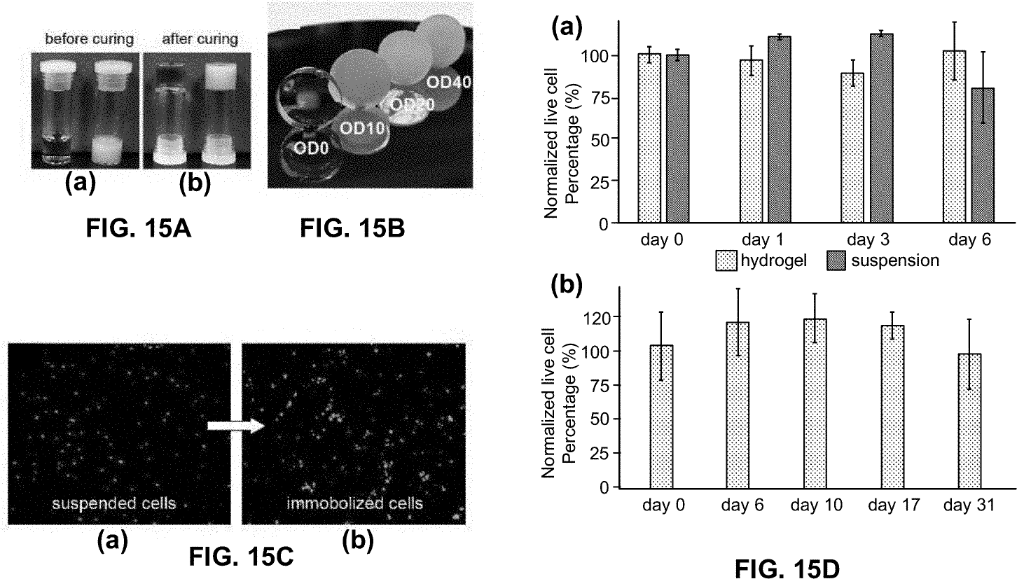

FIG. 15A are images showing an example of a polymeric hydrogel mixture before curing (part (a)) where the left vial is blank (e.g., no cells), and the right vial has a suspension of whole cells. Part (b) shows an example of polymeric hydrogel mixture after curing where the vials are upside-down, with the left vial is blank, (e.g., no cells), and the right vial has a cured suspension of whole cells.

In one aspect, the concentration of whole cells in the mixture may have a cell optical density (OD) in a range from about 4 to about 80. In exemplary approaches, the concentration of whole cells in the mixture may have an OD in a range of at least 20 to about 160. In other approaches, the concentration of whole cells in the mixture may have an OD in a range of about 30 to about 70. In yet other approaches, the concentration of whole cells in the mixture may have an OD in a range of about 20 to about 60. A particularly preferred formulation includes using M. capsulatus Bath cells in a concentration corresponding to about OD 40 in 12 wt. % acrylate-functionalized PEG pre-polymer (MW=20 kDa).

FIG. 15B is an image of cured hydrogel discs with whole cell concentrations of increasing optical density (OD), from OD 0 (e.g., no cells), OD 10, OD 20, and OD 40. In some approaches, density of whole cells in hydrogel mixture may be tuned to OD 160.

The viability of the whole cells in suspension compared to immobilized is visualized in the fluorescent microscopy images of FIG. 15C. Part (a) shows the fluorescent staining of all cells (bright cells) suspended in hydrogel. Part (b) shows the fluorescent staining of all cells after curing when the whole cells are immobilized in cured hydrogel. In both images, the darker stained cells (red fluorescence) are identified as dead cells. There is essentially no change in the number of dead cells in the suspended cells (part (a)) compared to the immobilized cells (part (b)).

Viability of the whole cells in suspension compared to cured hydrogel may be assessed by counting the fluorescent-stained cells. For example only, and not meant to be limiting in anyway, FIG. 15D shows the normalized live cell percentage assessed over time as shown in part (a) over one week, and longer, over one month in part (b). Over one week (e.g., 7 days), whole cells immobilized in cured hydrogel maintain their viability near 100% compared to whole cells in suspension, part (a). Without wishing to be bound by any theory, there may be essentially no difference in cell viability whether the cells are in suspension or immobilized in cured hydrogel. Looking to part (b) which illustrates the viability of whole cells immobilized in cured hydrogel for as long as a month, whole cells maintain a high level of viability through three weeks, and viability diminishes with a high level of variability at 31 days.

In various approaches, the pre-polymer of the mixture may be a monomer, macromer, etc. The concentration of pre-polymer of the encapsulation mixture (e.g., hydrogel material) may be in a range of about 10 wt. % to about 50 wt. % of the total weight of mixture. In some approaches, the concentration of pre-polymer may be about 10 wt. % to about 30 wt. % of total weight of mixture. In other approaches, the concentration of pre-polymer may be 20 wt. % to about 40 wt. % of total weight of the mixture. In some approaches, the concentration of pre-polymer may depend on the type of pre-polymer used.

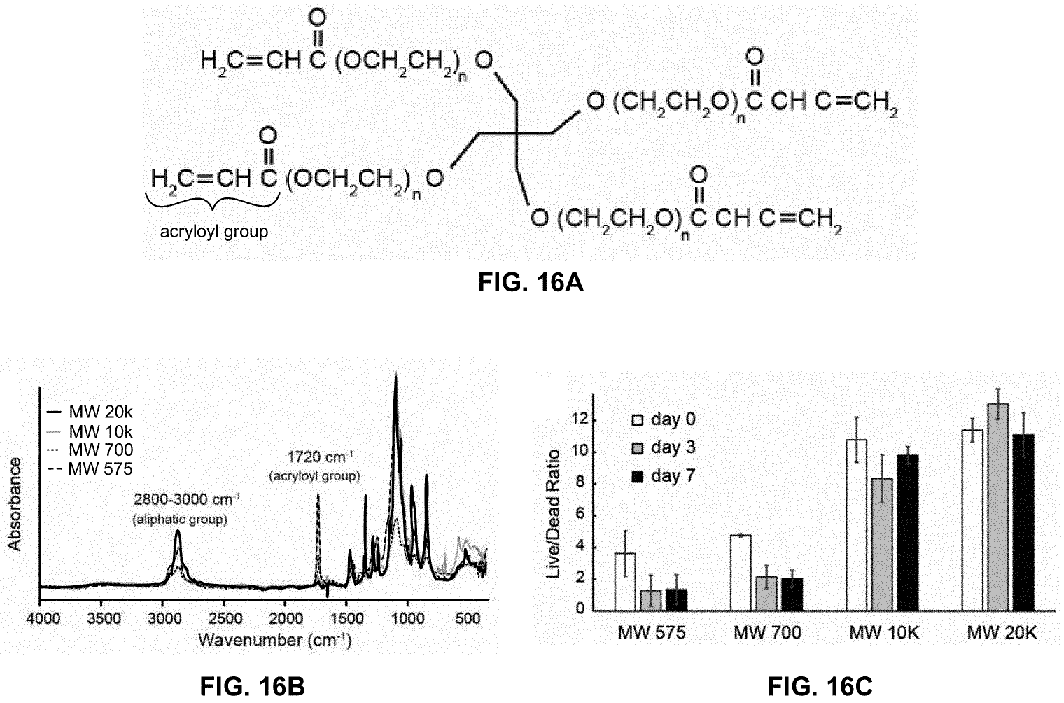

In one embodiment, the pre-polymer material in the encapsulant may include acrylate-functionalized polyethylene glycol (PEG) pre-polymer material. In one approach, the acrylate-functionalized PEG may include multiple acrylate groups. In a preferred approach, the acrylate-functionalized PEG may include more than two acrylate groups. Examples of exemplary pre-polymer material may include poly(ethylene) glycol (PEG) (e.g. acrylate-functionalized PEG such as PEGDA), gelatin, cellulose nanocrystals, alginate, N-siopropylacrylamide, amphiphilic silicones, etc. FIG. 16A illustrates a structure of a pre-polymer material PEGDA, where n is a number that extends the molecular weight of the pre-polymer.

Hydrogel compositions including lower molecular weight pre-polymers (e.g., 575 Daltons (Da) likely have a higher percentage of acryloyl groups (arrow on FIG. 16A) the composition of similar prepolymer concentration may include more 575 Da molecules. FIG. 16B is a plot of absorbance spectra of hydrogel compositions comprised of pre-polymers having different MWs. The 1720 peak shows the quantity of acryloyl bonds in the hydrogel composition, and, notably, the 575 Da PEGDA composition has a distinct peak for acryloyl bonds compared to the higher molecular weight PEGDAs, which do not exhibit a significant peak at 1720 thereby confirming that larger MW PEGDA molecules would have less of the acryloyl groups in the whole mixture. Without wishing to be bound by any theory, it is believed that an encapsulant hydrogel composition having reactive acryloyl groups (i.e., the acryloyl groups have not been completely cross-linked during curing, e.g., an incomplete curing reaction) may have a detrimental effect on the viability of the cells present in the encapsulant hydrogel composition

FIG. 16C presents an example of viability data of whole cells encapsulated with hydrogel compositions of different MW, e.g., 575 Da, 700 Da, 10K Da, and 20K Da, over a week (0, 3, and 7 days). For example only, and not meant to be limiting in any way, whole cells encapsulated with a hydrogel composition comprising lower MW pre-polymer (e.g., 575 and 700) demonstrate a lower ratio of live to cells, and the viability of the live cells may decline over time in these hydrogel compositions. Alternatively, whole cells encapsulated with hydrogel compositions having higher MW pre-polymer demonstrate a higher ratio of live to dead cells and live cells remain substantially viable over 7 days.

Thus, in some approaches, a pre-polymer hydrogel having a higher MW acrylate-functionalized PEG pre-polymer, e.g., having fewer total acryloyl groups in the hydrogel composition, may be a preferable pre-polymer for encapsulation of live cells. In one approach, a higher MW acrylate-functionalized PEG pre-polymer, e.g., greater than 700 Da, may be preferably for a whole cell encapsulant. In an exemplary approach, PEG-tetra-acrylate (PEGTA) pre-polymer may be included for encapsulation of live whole cells.

In another approach, if the reaction of UV curing of the pre-polymer hydrogel is allowed to run to completion, then the acryloyl groups in the 575 Da pre-polymer composition would become unreactive. Thus, without wishing to be to bound by any theory, it is believed that if the curing reaction is able to go to full completion, the hydrogel compositions having low MW pre-polymers may be useful as an encapsulant of whole cells.

In some approaches, the molecular weight of the pre-polymer may in a range of about 575 Daltons (Da) to about 100,000 Da but could be higher or lower. In one approach, a pre-polymer having a molecular weight of less than 575 Da tends to be less soluble and thus may be difficult to mix in the hydrogel composition. In some approaches, the molecular weight of the pre-polymer may be in a range of about 5000 Da to about 10,000 Da. In some approaches, the molecular weight of the pre-polymer may be in a range of about 10,000 Da to about 60,000 Da. In exemplary approaches, the molecular weight of the pre-polymer is in a range of about 10,000 Da to about 40,000 Da. In one exemplary approach, the pre-polymer includes acrylate-functionalized PEG (e.g., PEGDA, PEGTA, etc.) with a molecular weight in the range of 575 Da to 20,000 Da.

In various approaches, the whole cells may be mixed with the pre-polymer formulations and a photoinitiator. In some approaches, an exemplary example of photoinitiator may be lithium phenyl-2,4,6-trimethylbenzoylphosphinate (LAP).

In one aspect, the mixture of whole cells, pre-polymer, and photoinitiator may be cured by UV radiation for crosslinking the pre-polymer. In some approaches, the curing may include radiation with UV (at a range of 300 nm to 450 nm) for a duration of time effective for crosslinking the pre-polymer for encapsulating the whole cells. In some approaches, the curing with UV radiation may occur for a duration of under approximately 30 seconds. In other approaches, the curing with UV radiation may occur for a duration of under 15 seconds. In other approaches, the curing with UV radiation may occur for a duration of under 10 seconds.

In one aspect, a product includes a structure having a plurality of whole cells encapsulated in a polymer, where the polymer is cross-linked. In some approaches the structure may be a polymeric network encapsulating a plurality of whole cells. In various approaches, the concentration of pre-polymer in the mixture may equal the concentration of cross-linked polymer encapsulating the whole cells. In some approaches, the curing may not change the amount of pre-polymer originally added to the mixture.

In some approaches, the components 302 of the membrane may include a plurality of enzymatic reactive components having one or more of: isolated enzymes, transmembrane enzymes, cell-membrane-bound enzymes, liposomes coupled to/comprising an enzyme, etc.

In some exemplary approaches, a plurality of whole cells converts methane to methanol. In some approaches, enzymatic reactive components may convert methane to methanol. For example, but are not limited to, enzymatic reactive components may include formate dehydrogenase, carbonic anhydrase, cytochrome p450, hydrogenase, particulate methane monooxygenase (pMMO), photosynthetic complexes, etc. In various approaches, a plurality of whole cells may convert methane to methanol better than enzymatic reactive components because the whole cell includes all cofactors and processes for the metabolic pathway. In contrast, enzymatic reactive components may need co-factors and various additives to function and convert methane to methanol.

Moreover, while in particular approaches, the components 302 of the membrane 300 may include enzymatic reactive components and whole cells, and in some of these approaches, the enzymatic reactive components may be the same (e.g., comprise the same structure and/or composition); other of these approaches the components 302 may include at least two of the enzymatic reactive components and/or whole cells to be different from one another (e.g., have a different structure and/or composition, be of different species or strains, etc. as would be appreciated by a person having ordinary skill in the art upon reading the present disclosures).

In approaches where at least one of the enzymatic reactive components includes a membrane-bound enzyme, said enzyme may be stabilized prior to incorporation into the polymer network 304. For instance, in one stabilization approach, cell fragments comprising the enzyme of interest may be used, and directly incorporated into the polymer network 304. In another stabilization approach, a lipopolymer may first be formed by linking a lipid to a polymer of interest. The lipid region of the polymer may spontaneously insert into the cell membrane, thereby creating a polymer functionalized liposome, which may be incorporated in the polymer network 304. In yet another stabilization approach, the enzyme of interest may be coupled to and/or encapsulated into a nano-lipo-protein particle (NLP), which may then be incorporated in the polymer network 304.

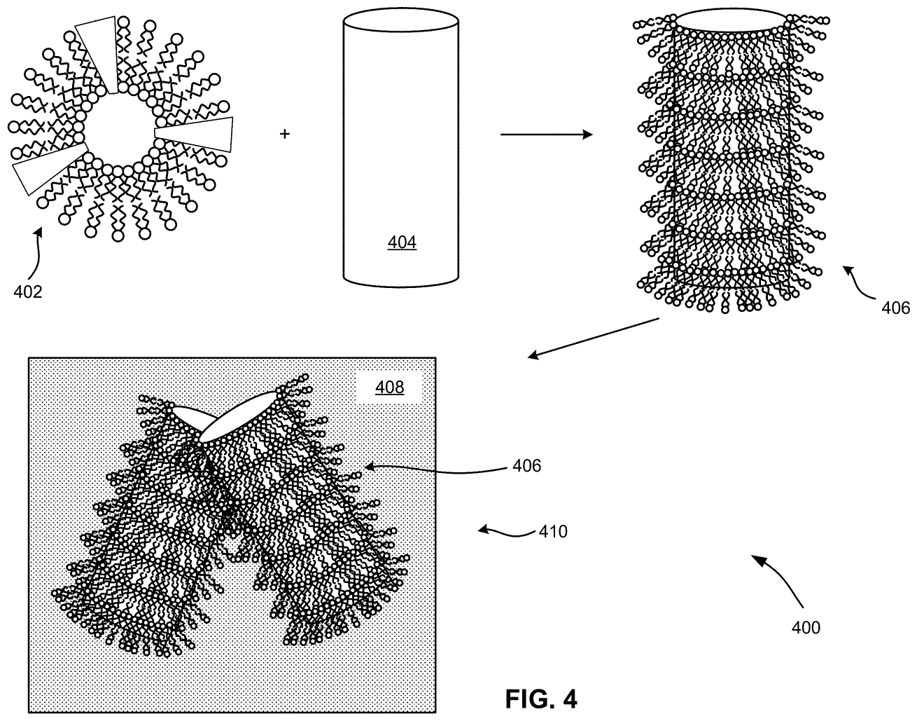

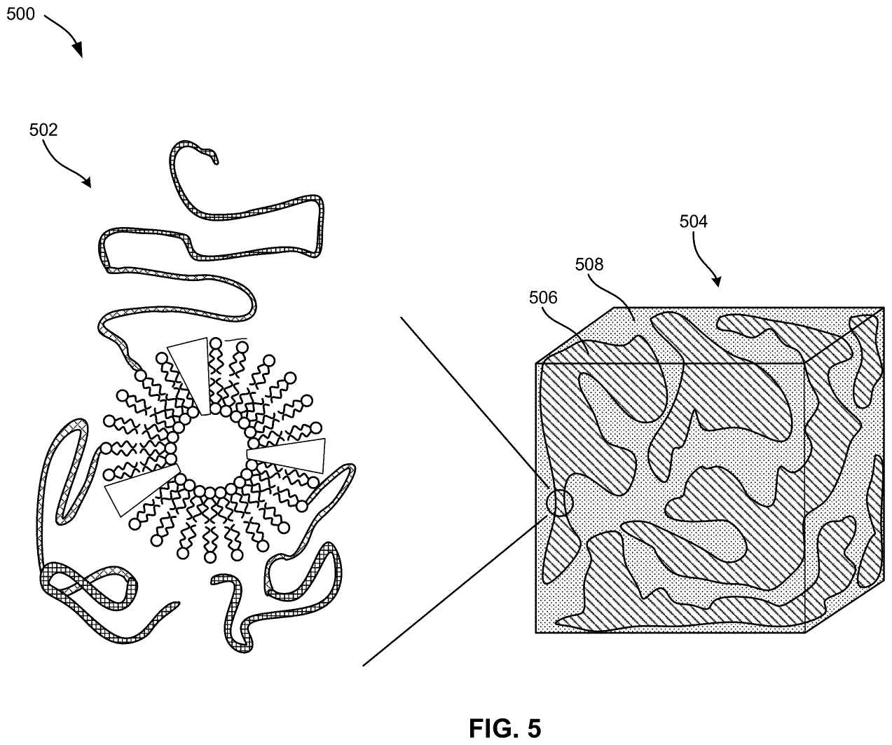

The components 302 such as enzymatic reactive components and/or whole cells may be incorporated into the polymeric network 304 via several methods including, but not limited to: attaching the components, e.g., enzymatic reactive components and/or whole cells, to electrospun fibers of a first polymer, and backfilling with a second polymer (see, e.g., the method described in FIG. 4); directly incorporating the components 302 e.g., enzymatic reactive components and/or whole cells into a polymer or block-copolymer network before or after crosslinking the network (see, e.g., the method described in FIG. 5); and other suitable incorporation methods as would become apparent to one having skill in the art upon reading the present disclosure.

With continued reference to FIG. 3, the polymeric network 304 may include at least a two phase polymer network, e.g. a polymer network comprising two or more polymeric materials. This polymer network 304 may be configured to serve as a mechanical support for the components 302 e.g., enzymatic reactive components and/or whole cells, embedded therein, concentrate reactants, and remove products. In preferred approaches, the polymeric network 304 may include nanometer scale domains of higher reactant permeability, as well as nanometer scale domains of higher product permeability.

In particular approaches involving gas to liquid reactions, the polymeric network may include nanometer scale domains of higher gas permeability, such as silicon, as well as nanometer scale domains of higher product permeability, such as a polyethylene glycol (PEG) based hydrogel. These domains of high gas permeability typically also have higher gas solubility, increasing the local concentration of reactants (e.g., relative to the aqueous medium in a stirred-tank reactor) and therefore increase the turnover frequency of the components 302 e.g., enzymatic reactive components and/or whole cells; whereas, the domains of low gas permeability and high product permeability may efficiently remove the product and reduce product inhibition (thereby also increasing the turnover frequency and stability of the components 302 e.g., enzymatic reactive components and/or whole cells) or serve to stabilize the enzymatic reactive components. In various approaches, the permeability for the “higher gas permeability phase” may be greater than 100 barrer.

In some approaches, the polymer network 304 may comprises a di-block copolymer network. In other approaches, the polymer network 304 may include a tri-block copolymer network. Suitable polymers for the polymeric network 304 may include silicone polymers, polydimethylsiloxane (PDMS), poly(2-methyl-2-oxazoline) (PMOXA), polyimide, PEG, acrylate-functionalized PEG, (e.g., polyethylene glycol diacrylate (PEGDA), polyethylene glycol tetra-acrylate (PEGTA), etc.), poly(lactic acid) (PLA), polyvinyl alcohol (PVA), and other such polymers compatible with membrane proteins and block copolymer synthesis as would become apparent to one skilled in the art upon reading the present disclosure.

In more approaches, each pre-polymer in the polymeric network 304 may have a molecular weight ranging from about 500 Da to about 500 kDa(kDa), more preferably ranging from about 500 Da to about 20 kDa, and most preferably ranging from about 575 Da to about 20 kDa. Moreover, in various approaches the pre-polymers may be present in an amount ranging from about 10 wt. % to about 50 wt. %.

In other approaches, the polymeric network 304 may include a mixture of at least one pre-polymer material and at least one inorganic material.

In various approaches, a thickness, t₁, of the enzyme embedded polymer network 304 may be in a range from about 1 micrometer to about 2 millimeters.

As indicated above, the membrane 300 may be configured to separate the reactants and products associated with a catalyzed reaction of interest. In various approaches, the membrane 300 may provide sufficient surface area on a first side 310 for contacting fluids to support efficient transport of reactants to and from reacting components 302, e.g. enzymatic reactive components and/or whole cells. In some approaches, the separating by the membrane may include being configured to be a barrier to the products formed from the reacting components 302 in the membrane 300. For example, in some approaches, reactants may be permeable at the first layer, e.g. a reactant permeable polymer layer 306 of the membrane 300 but impermeable at the second layer, e.g. a product permeable polymer layer 308 thereby allowing reactant to enter and exit the polymeric network 304 from the reactant permeable polymer layer 306. In some approaches, the product permeable polymer layer 308 of the membrane may be a barrier to a reactant.

Furthermore, products formed from the reactants may be permeable at the product permeable polymer layer 308 of the membrane but impermeable at the reactant permeable polymer layer 306 thereby allowing products to exit the polymeric network 304 from the product permeable polymer layer 308 but not the first layer 306. In some approaches, the reactant permeable polymer layer 306 of the membrane may be a barrier to a product.

In various approaches, reactants and products may be two different fluids, such as liquids and gasses, aqueous species and non-aqueous species, polar species and non-polar species, etc. In some approaches, the membrane 300 comprises a polymeric network 304 configured to separate a first fluid and a second fluid, where the first and second fluids are different.

In one exemplary approach where the membrane 300 may be configured to separate methane and oxygen from methanol. The reactant permeable polymer layer 306 of the membrane is permeable to methane (e.g. the reactant) thereby allowing methane to enter the polymeric network 304 of the membrane 300. The reactive components 302 of the polymeric network 304 catalyze methane oxidation to form the product methanol in the following reaction in Equation 1.

CH₄+O₂→CH₃OH+H₂O

Reactant→Product Equation 1

In one exemplary example, the product permeable polymer layer 308 of the membrane 300 is configured to be impermeable to the reactant methane (CH₄), so any residual methane (e.g. reactant) may exit the polymeric network 304 via only the reactant permeable polymer layer 306. The membrane 300 may act as a barrier to methane passing from the first side 310 of the membrane 300 at the reactant permeable polymer layer 306 through to the second side 312 of the membrane 300 at the product permeable polymer layer 308.

Furthermore, the product permeable polymer layer 308 of the membrane is configured to be permeable to the products methanol (CH₃OH) and water (H₂O), but the reactant permeable polymer layer 306 is configured to be impermeable to methanol and water, so the products may only exit via the product permeable polymer layer 308 of the membrane 300. The membrane 300 may act as a barrier to products methanol and water passing from the second side 312 of the membrane 300 at the product permeable polymer layer 308 through to the first side 310 of the membrane 300 at the reactant permeable polymer layer 306.

In some approaches, the methane reactant concentration may be in a range from about 1 to about 100 mM, the oxygen reactant concentration may be in a range from about 1 to about 100 mL, and the methanol product concentration range may be in a range from about 0.1 to about 1000 mM.

To further facilitate reactant-production separation, at least a portion of one surface of the membrane 300 may include an optional reactant permeable polymer layer 306 coupled thereto, as shown in FIG. 3. In preferred approaches, this reactant permeable polymer layer 306 may also be impermeable to products generated from the reactions catalyzed by the components 302 e.g., enzymatic reactive components and/or whole cells. Suitable polymeric materials for this reactant permeable polymer layer 306 may include, but are not limited to, nanofiltration, reverse-osmosis, or chemically selective membranes, such as poly(ethylene imine), PVA, poly(ether ketone) (PEEK), cellulose acetate, or polypropylene (PP).

In some approaches, a thickness, t₂, of the reactant permeable polymer layer 306 may be in a range from about 0.1 to about 50 micrometers. This optional reactant permeable polymer layer 306 may be particularly suited for approaches involving an organic polar reactant and an organic non-polar product (and vice versa).

As also shown in FIG. 3, at least a portion of one surface of the membrane 300 may include an optional product permeable polymer layer 308 coupled thereto. This product permeable polymer layer 308 may preferably be coupled to a surface of the membrane 300 opposite that on which the reactant permeable polymer layer 306 is coupled, thereby facilitating entry of reactants (e.g., gaseous reactants) on one side of the membrane 300, and removal of the reaction products (e.g., liquid reaction products) on the opposing side of the membrane 300. In more preferred approaches, this product permeable polymer layer 308 may also be impermeable to the reactants introduced into the enzyme embedded polymer network 304. Suitable polymeric materials for this product permeable polymer layer 308 may include, but are not limited to, nanofiltration, reverse-osmosis, or chemically selective membranes, such as poly(ethylene imine), PVA, poly(ether ether ketone) (PEEK), cellulose acetate, or polypropylene (PP). In some approaches, a thickness, t₃, of the product permeable polymer layer 308 may be in a range from about 0.1 to about 50 micrometers.

In some approaches, a cofactor may be included for one or more of the enzymatic reactive components to function. Accordingly, cofactors may be supplied by co-localized enzymes in reactor domains of the polymer network 304 (not shown in FIG. 3), and/or be retained within a cofactor impermeable layer coupled to a portion of the membrane 300 (not shown in FIG. 3). However, and particularly in the case of whole cells, cofactors may not need to be included, in various aspects. Advantageously, avoiding the need to provide cofactors significantly reduces the cost of utilization and enables performing the various bioreactions (whether carbon capture, ethanol production, etc.) in a scalable manner.

In various approaches, a total thickness, t₄, of the membrane 300 may be in a range from about 10 to about 3100 micrometers.

In yet more approaches, the membrane 300 may be shaped into features, structures, configurations, etc. that provide a desired surface area to support efficient transport of reactants to, and products from, the components 302, e.g., enzymatic reactive components and/or whole cells. For instance, the membrane 300 may be shaped into at least one of: a hollow fiber membrane, a micro-capsule membrane, a hollow tube membrane, a spiral wound membrane, etc.

Advantageously, and regardless of the particular application to which the inventive systems and techniques may be applied, the need for seeding cells or enzymatic reactive components is eliminated, since whole, live cells may be encapsulated within the scaffold itself.

Referring now to FIG. 4, a method 400 for embedding enzymatic reactive components within a two phase (AB) polymer network is shown according to one aspect. As an option, the present method 400 may be implemented in conjunction with features from any other aspect listed herein, such as those described with reference to the other FIGS. Of course, this method 400 and others presented herein may be used to form structures for a wide variety of devices and/or purposes, which may or may not be related to the illustrative aspects listed herein. It should be noted that the method 400 may include more or less steps than those described and/or illustrated in FIG. 4, according to various aspects. It should also be noted that that the method 500 may be carried out in any desired environment.

As shown in FIG. 4, an enzymatic reactive component and/or whole cells 402 is/are adsorbed to at least one portion of the exterior surface of polymer A 404, thereby forming enzyme-embedded polymer A 406. In preferred approaches, polymer A 404 may comprise one or more hydrophobic, reactant permeable (e.g., gas permeable) polymeric materials configured to provide high concentrations and fast transport of reactants. In further approaches, polymer A 404 may be a polymer nanofiber generated using electrospinning, extrusion, self-assembly, or other suitable technique as would become apparent to one skilled in the art upon reading the present disclosure. In additional approaches, such a polymer A nanofiber may be crosslinked to other polymer A nanofibers. In one exemplary approach, polymer A 404 comprises PDMS.

In various approaches, the enzymatic reactive component and/or whole cells 402 may be selected from the following group: an isolated enzyme, an enzyme comprising a cell fragment (e.g., a cell membrane or cell membrane fragment), and a liposome comprising/coupled to an enzyme. In some approaches, the enzymatic reactive component and/or whole cells 402 may include at least one of: formate dehydrogenase, carbonic anhydrase, cytochrome p450, hydrogenase, particulate methane monooxygenase (pMMO), photosynthetic complexes, etc. In still more approaches, the enzymatic reactive component and/or whole cells 402 may include whole, wet or dry cells of any organism described herein and/or as would be appreciated as suitable by a person having ordinary skill in the art upon reading the present descriptions.

In the non-limiting aspect shown in FIG. 4, a plurality of enzymatic reactive components and/or whole cells 402 may be adsorbed to one or more portions of the exterior surface of polymer A 404. These enzymatic reactive components and/or whole cells 402 may be adsorbed to at least the majority, or more preferably about an entirety, of the exterior surface of polymer A 404. The lipid bilayer vesicles of the enzymatic reactive components and/or whole cells 402 may spontaneously collapse on the exterior surface of polymer A 404, thereby forming a lipid-bilayer functionalized surface.

As further shown in FIG. 4, the enzyme-embedded polymer A 406 may be mixed with polymer B 408 to create the two phase (AB) polymer monolith 410 with the enzymatic reactive components and/or whole cells 402 at the interface between the two phases. In preferred approaches, polymer B 408 may comprise one or more hydrophilic, product permeable polymeric materials configured to provide transport of products, as well as stabilize the enzymatic reactive components and/or whole cells 402. For instance, in one specific approach, polymer B 408 may be a hydrophobic polymer hydrogel.

While the resulting polymeric network shown in FIG. 4 includes two phases (i.e., polymer A and polymer B), it is important to note that said polymeric network may include more than two phases in additional approaches.

Referring now to FIG. 5, a method 500 for embedding enzyme reactive components within a two phase (AB) polymer network is shown according to another aspect. As an option, the present method 500 may be implemented in conjunction with features from any other aspect listed herein, such as those described with reference to the other FIGS. Of course, this method 500 and others presented herein may be used to form structures for a wide variety of devices and/or purposes, which may or may not be related to the illustrative aspects listed herein. It should be noted that the method 500 may include more or less steps than those described and/or illustrated in FIG. 5, according to various aspects. It should also be noted that that the method 500 may be carried out in any desired environment.

As shown in FIG. 5, enzymatic reactive components and/or whole cells 502 may be directly incorporated in a block copolymer network 504 prior to or after cross-linking said network. As described herein, each enzymatic reactive component and/or whole cell 502 may be independently selected from the following: an isolated enzyme, an enzyme comprising a cell fragment (e.g., a cell membrane or cell membrane fragment), and a liposome comprising/coupled to an enzyme; optionally where including whole cells, enzymatic reactive components and/or whole cells 502 may include whole cells of any organism described herein or as would be understood as suitable by a person having ordinary skill in the art upon reading the present disclosure. In some approaches, the enzymatic reactive component and/or whole cells 502 may include at least one of: formate dehydrogenase, carbonic anhydrase, cytochrome p450, hydrogenase, particulate methane monooxygenase (pMMO), photosynthetic complexes, etc., and optionally may include whole cells of any organism described herein or as would be understood as suitable by a person having ordinary skill in the art upon reading the present disclosure.

As shown in the non-limiting aspect of FIG. 5, the block copolymer network 504 is a di-block copolymer network comprising two different polymers (polymer A 506 and polymer B 508). In preferred approaches, polymer A 506 may comprise one or more reactant permeable, hydrophobic polymeric materials, whereas polymer B 508 may comprise one or more product permeable, hydrophilic polymeric materials. It is again important to note that while the block copolymer network 504 shown in FIG. 5 includes two phases (i.e., polymer A 506 and polymer B 508), said block copolymer network may include more than two phases in other approaches.

In various approaches, the enzymatic reactive components and/or whole cells 502 may be incorporated directly into the block copolymer network 504 using lipopolymers (preferably di-block lipopolymers). Lipopolymers may be generated by linking a lipid to a polymer of interest, such as PEG, creating PEG-lipid conjugates, such as PEG-phosphatidylethanolamie. The lipid region of the polymer may spontaneously insert into the cell membrane, thereby creating a polymer functionalized liposome.

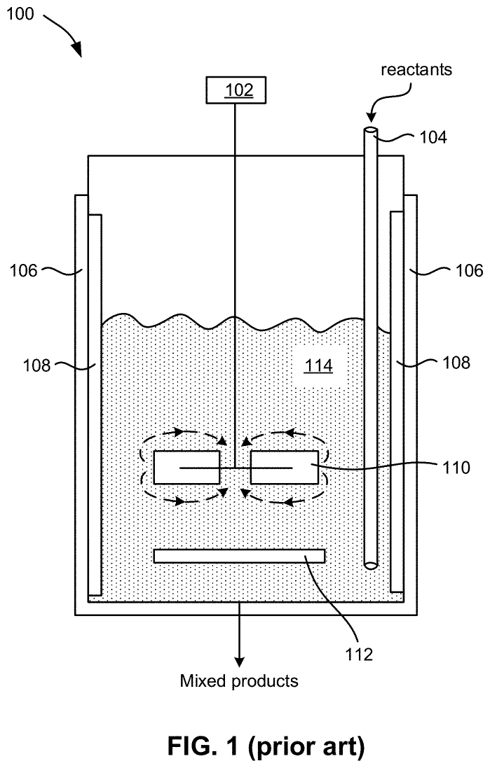

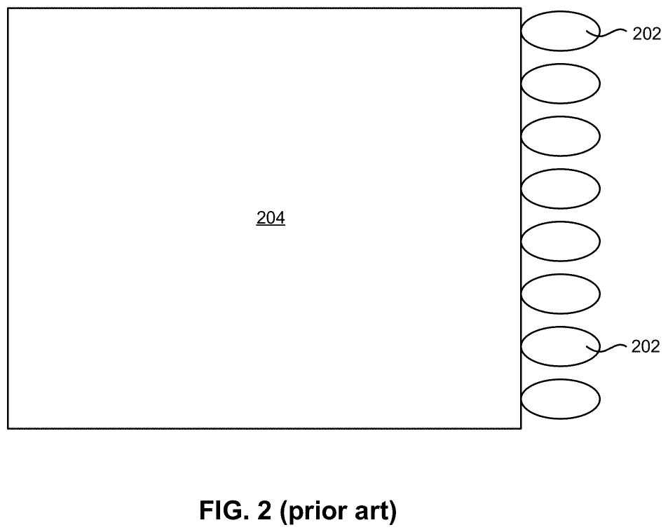

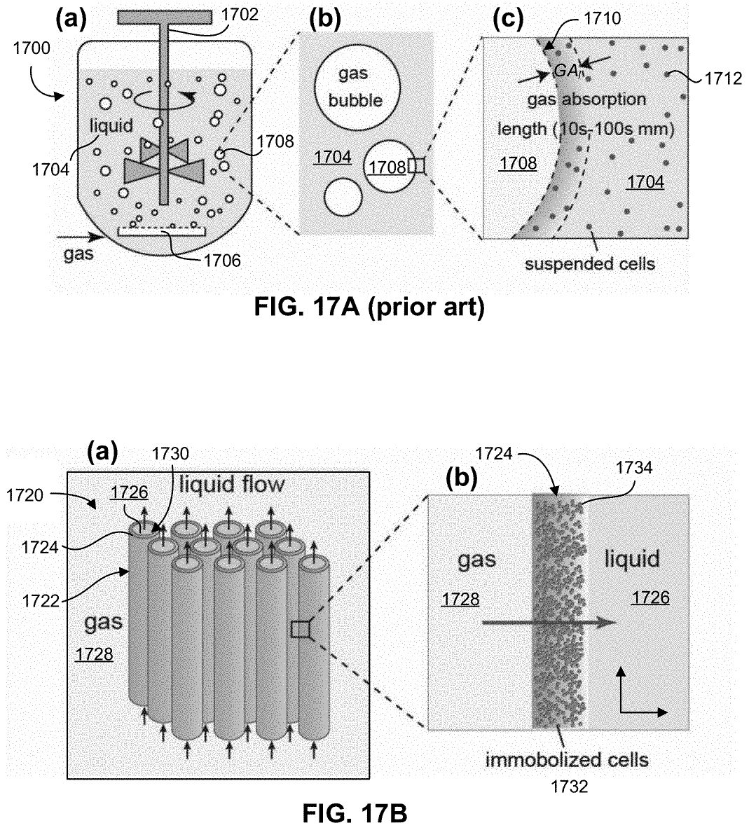

As shown in FIG. 17A, a conventional stirred-tank reactor 1700 (part (a) and described in detail in FIG. 1) relies on an agitator 1702 (e.g., stirrer), in a liquid aqueous medium 1704 with gas bubbling from a sparger 1706 at the bottom of the aqueous medium 1704. Gas exchange occurs at the gas bubbles 1708 created from the sparger 1706 in the aqueous medium 1704. Part (b) illustrates a magnified view of the gas bubbles 1708 in the aqueous medium 1704. Part (c) illustrates a further magnified view of part (b) showing the surface 1710 of the gas bubble 1708 where the suspended cells 1712 in the aqueous medium 1704 interact with the gas bubble 1708. The area along the outer surface 1710 of the gas bubble 1708 has a gas absorption length (GAₗ) that is typically in the 10s to 100s of millimeters (mm).

The conventional stirred-tank reactors 1700 tend to be energy inefficient as well as low levels of mass transfer due to the disparate interactions of the suspended cells 1712 in the aqueous medium 1704 and the interaction with the gas bubbles. It would be desirable to increase the mass transfer of gas absorption density of the suspended cells.

FIG. 17B illustrates a schematic drawing of a bioreactor 1720 that is a 3D structure 1722 configured to optimize mass transfer of gas absorption, according to one embodiment. In one approach the bioreactor may be a single 3D structure 1722 scaled to a large size. In another approach, the bioreactor 1720 may be a plurality of 3D structures 1722. In one approach, the 3D structures may have a geometric shape defined by the application. In various approaches, the 3D structure may be printed into a 3D shape, e.g., hollow cylinder, lattice, cube, etc. In one approach, the 3D structure 1722 may have a cylinder shape. In one approach, the 3D structure (e.g., cylinder) may have a vertical orientation. In other approaches, the 3D structure may have a horizontal orientation. In yet other approaches, the 3D structure may have an orientation preferred by the configuration of the application (e.g., a bioreactor). The 3D structure 1722 may be formed as a hollow structure having a wall 1724 comprising the encapsulated cells.

According to one embodiment, the 3D structures 1722 are hollow tubes positioned vertically in the gas 1728 with liquid 1726 flowing in a vertical direction through the hollow portion of the 3D structure 1722.

Part (b) is a magnified view of the mass transfer of the gas 1728 to the liquid 1726 through the wall 1724 of the 3D structure 1722. The gas 1728 absorbs through the wall 1724 of the 3D structure toward the hollow portion 1730 of the 3D structure in a direction about orthogonal to the vertical direction of the flow of the liquid 1726. The wall 1724 of the 3D structure 1722 includes immobilized cells 1732 in a cured hydrogel 1734, according to one approach.

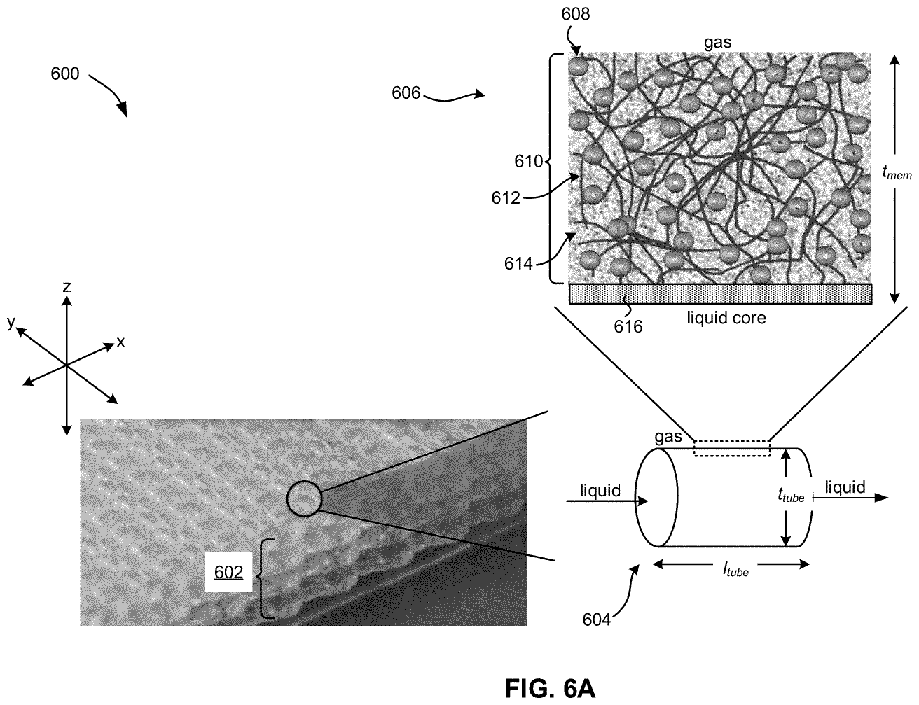

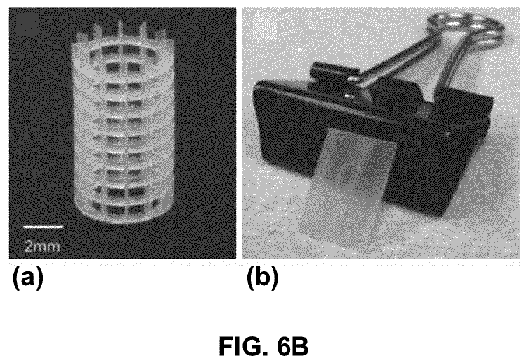

Referring now to FIG. 6A, a bioreactor 600 comprising a network/lattice of 3D structures configured to optimize mass transfer is shown according to one aspect. As an option, the bioreactor 600 may be implemented in conjunction with features from any other aspect listed herein, such as those described with reference to the other FIGS. Of course, the bioreactor 600 and others presented herein may be used in various applications and/or in permutations which may or may not be specifically described in the illustrative aspects listed herein. For instance, the bioreactor 600 may be used in any desired environment and/or include more or less features, layers, etc. than those specifically described in FIG. 6A.

In one aspect, a bioreactor may include a 3D structure where the 3D structure includes a gas-permeable material and polymer-encapsulated whole cells. In one approach, at least one side (e.g., wall, edge, border, etc.) of the 3D structure is infilled with the polymer-encapsulated whole cells. In one aspect, a side of a 3D structure is gas permeable. In other approaches, the side may be comprised of material that is permeable to gas. The material may have holes, spaces, pores, etc. and/or the structure may have holes, spaces, pores, etc.

In some approaches, the 3D structure may be a printed 3D structure. In one approach, the printed 3D structure may be a lattice. The lattices may be, in one approach, composed of a silicone polymer, and the geometry and lattice structure may be easily modified. In some approaches, the wall may have space between a lattice pattern that is permeable to gas.

In various approaches, the polymer formulation may be printed in different geometries. According to one aspect, a lattice may be with PμSL is shown in part (a) of FIG. 6B. The lattice may be designed to be a hollow tube structure with the walls infilled with the polymer-cell solution and then cured UV radiation with the center of the tube remaining hollow.

As particularly shown in FIG. 6A, the bioreactor 600 includes a network/lattice 602 of 3D structures. In some approaches, the network/lattice 602 includes multiple layers (e.g., 2, 3, 4, 5, 6, 7, or more layers, etc.) of 3D hollow tubes 604. It is important to note, however, that the hollow tube network/lattice 602 of the bioreactor 600, and others disclosed herein, may include one or more layers of 3D hollow tubes 604 in various approaches. The hollow tubes 604 may preferably be oriented in the lattice such that their hollow interiors are perpendicular to a thickness direction of the lattice (e.g., perpendicular to the z axis shown in FIG. 6A). In some approaches, the printed 3D structure is a tube, where a wall of the tube may be gas-permeable and an inner surface of the wall defining a center portion of the tube.

The lattice as shown in part (a) of FIG. 6B may be suitable for use with methanotroph cells. In another aspect, a lattice mesh created with a DIW printing technique is shown in FIG. 6B part (b). The silicone structure as shown in part (b) may be suitable for use with methanotroph cells.

In some approaches, the bioreactor may include a buffer in the center portion of the tube, where the buffer comprises nutrients for the polymer-encapsulated whole cells. In various approaches, the polymer-encapsulated whole cells may include living whole cells that have a characteristic to remain viable in the bioreactor (e.g., cured infill of the 3D structure) for a duration of at least five days. In some approaches, the whole cells may remain viable in the bioreactor for a duration of at least 6 days, at least 7 days, at least 8 days, etc. In some approaches, the viability of the whole cells in the bioreactor may depend on the type of whole cell encapsulated in the bioreactor.

In some approaches, the buffer may be changed periodically (e.g., every day, every 3 days, every 5 days, every 7 days, etc.) with fresh nutrients to extend the viability of the whole cells encapsulated in the polymer of the bioreactor.

In various approaches, the polymer-encapsulated whole cell formulation described herein may be cured within structure lattices that were made with PμSL or DIW technology. In some approaches, the curing of the polymer-encapsulated whole cells allows the polymeric network of whole cells to infill the spaces of the lattice structure.

Referring again to FIG. 6A, in some approaches, the bioreactor 600 may have a thickness (as measured parallel to the z-axis in FIG. 6A) in a range from about 1 to about 300 cm, and a length (as measured in a direction parallel to the y-axis of FIG. 6A) and width (as measured in a direction parallel to the x-axis of FIG. 6A) scaled to the application, ranging from about 2 cm for laboratory applications to 10 meters for industrial applications.

The walls of each hollow tube 604 may comprise a membrane material 606, such as the membrane material of FIG. 3, configured to separate reactants (e.g., gaseous reactants) and products (e.g., hydrophilic products). Accordingly, the hollow tubes 604 form polymer microchannels through which the hydrophilic reaction products may flow.

As particularly shown in FIG. 6A, the membrane material 606 of each hollow tube 604 may comprise a plurality of enzymatic reactive components and/or whole cells 608 (e.g., isolated enzymes, membrane-bound enzymes, liposomes comprising/couple to an enzyme, etc.) embedded throughout a polymer network 610. The polymer network 610 may comprise reactant permeable fibrils of a first polymer 612 that increase the local concentration of reactants and enhance mass transfer throughout the membrane material 606. In some approaches, the enzymatic reactive components and/or whole cells 608 may be immobilized on the fibrils of the first polymer 612. The polymer network 610 may also include at least another polymer material (e.g., a hydrogel matrix material) configured to hydrate the enzymatic reactive components and/or whole cells 608 and provide a route for hydrophilic product removal. The membrane material 606 may also include an optional reactant permeable (product impermeable) layer 614 coupled to one side (e.g., an exterior side) of the polymer network 610 and/or a product permeable (reactant impermeable) layer 616 coupled to the opposite side (e.g., an interior side) of the polymer network 610. The optional product permeable (reactant impermeable) layer 616 may also facilitate product removal and prevent coenzyme and/or cofactor diffusion into the liquid core that contains the desired products.

The thickness, tₘₑₘ, of the membrane material 606 may be in a range from about 10 to about 1000 micrometers. In some approaches, tₘ may be about 300 μm. Additionally, the thickness, t_{tube}, of each hollow tube 604 may be in a range from about 10 micrometers to about 10 millimeters. In various approaches, t_{tube} may be about 1 mm. In yet more approaches, the length, l_{tube}, of each hollow tube 604 may be in a range from about 5 centimeters to about 10 meters.

It is important to note that while the cross section of each hollow tube 604, as taken perpendicular to the y-axis of FIG. 6A, is shown a circular, this need not be the case. For instance, in other approaches, each hollow tube 604 may have a cross sectional shape that is elliptical, rectangular, square, triangular, irregular shaped, etc. Moreover, in preferred approaches, each hollow tube 604 may have the same cross sectional shape, materials, and/or dimensions; however, this again need not be case. For instance, in alternative approaches, at least one of the hollow tubes 604 may have a cross sectional shape, materials, and/or dimensions that are different than that of another of the hollow tubes 604.

In one particular approach, one or more of the hollow tubes 604 in at least one of the layers may differ from one or more hollow tubes 604 in at least another of the layers with respect to: cross sectional shape, and/or one or more membrane material(s), and/or one or more dimensions. In another particular approach, one or more of the hollow tubes 604 in at least one of the layers may differ from at least another hollow tube 604 in the same layer with respect to: cross sectional shape, and/or one or more membrane materials, and/or one or more dimensions.

In yet further approaches, the spacing between the hollow tubes 604 in at least one of the layers may be about uniform. In more approaches, the spacing between the hollow tubes 604 in at least one of the layers may vary throughout the layer. For example, in one such approach, at least one of the layers may have at least one area having an average spacing, s₁, between adjacent hollow tubes 604, and at least a second area having an average spacing s₂, where s₁ and s₂ are different. In yet other approaches, the spacing between the hollow tubes 604 in at least one of the layers may differ from the spacing between the hollow tubes 604 of at least another of the layers.

Where a bioreactor 600 include whole cells 608, preferably the bioreactor may also include additional components such as gelatin, cellulose nanocrystals, acrylate-functionalized PEG, etc. as described in greater detail herein and/or as would be appreciated by a person having ordinary skill in the art upon reading the present descriptions.

In summary, the presently disclosed inventive concepts include, but are not limited to, formulations of polymer and whole cells that can be UV cured within a 3D printed scaffold or used as ink to directly print additively manufactured whole cell bioreactors. The ability to use the formulation with various additive manufacturing techniques the geometry of the structure to be defined and controlled. The methods described herein may overcome mass transfer limitations inherent to conventional stirred-tank reactors. Additionally, the cells remain alive and consume reactant over multiple days. By incorporating the whole cell, the catalysis may result in the production of valuable chemical products without the need for an expensive cofactor.

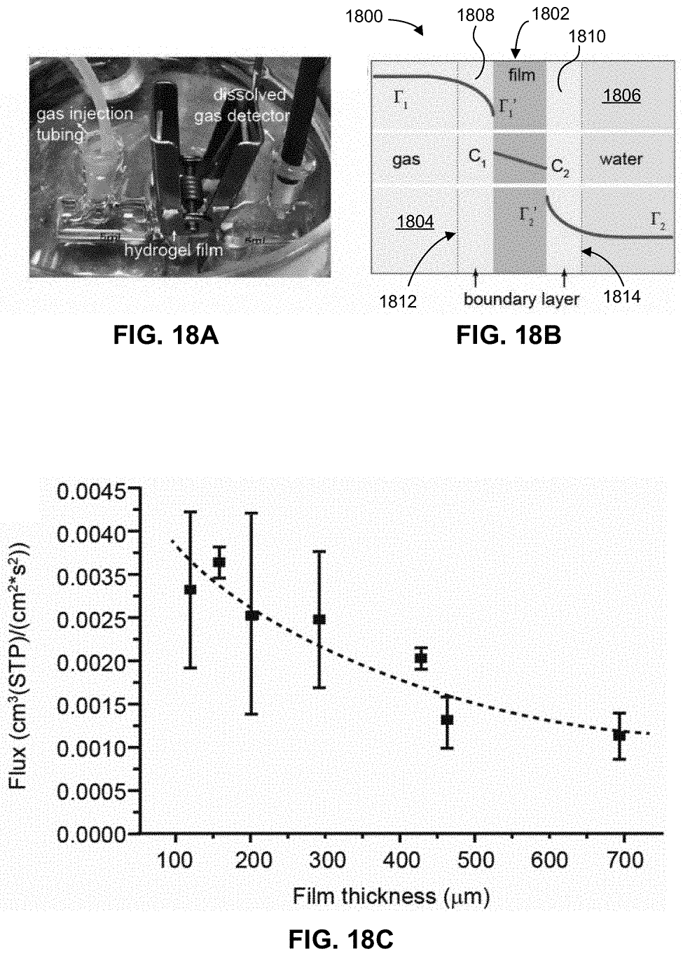

An example of the permeability of a hydrogel film is shown in FIGS. 18A-18C. The measurements for permeability of the film may be measured using a permeability cell positioned in a water bath as shown in the image of FIG. 18A and the schematic drawing of FIG. 18B. A gas is injected into the system by the gas injection tubing of the apparatus shown in FIG. 18A. The dissolved gas that crosses the hydrogel film is detected by a gas detector. The system may determine the permeability of gas through a thin film of the material, where dissolved CO₂ permeating through one side of a film to the other side where the film may be measured for CO₂ transport depending on thickness of the film.

FIG. 18B shows the concentration profile 1800 across the hydrogel film 1802 sandwiched between a gas 1804 and water 1806. As shown, boundary layers 1808, 1810 will form at the gas-hydrogel interface 1812 and at the hydrogel-water interface 1814, respectively. The concentration of gas 1804 varies in each component. For example, for a system of CO₂ as the gas, bulk CO₂ concentrations (designated Γₙ) may be measured in the gas 1804 portion (Γ₁) and the water 1806 portion (Γ₂). Interface CO₂ concentrations (designated Γ′ₙ) occur at the gas-hydrogel interface 1812 (Γ′₁) and at the hydrogel-water interface 1814 (Γ′₂). The CO₂ concentration of the hydrogel 1802 (designated Cₙ) may be measured at each boundary 1808 (C₁) and 1810 (C₂).

FIG. 18C is a plot of the flux of dissolved CO₂ across the hydrogel membrane as a function of membrane thickness. As illustrated in FIG. 18B, the flux may be calculated from a measured change in CO₂ partial pressure across the membrane. The plot of FIG. 18C shows that at membrane thicknesses of 100 and 200 micron (μm), there is efficient flux of CO₂ across the membrane. Moreover, the plot shows sufficient evidence that membrane thicknesses in the 10s of microns range would increase flux gas across the membrane.

In some approaches, a thickness of hydrogel membrane may be in a range of about 10 μm to about 5000 μm (5 mm). In some approaches, the flux of gas at the interface of the membrane and the gas is independent of the overall thickness of the hydrogel membrane, thus thicknesses of a hydrogel membrane comprising encapsulated cells above 500 μm may not have a significant effect on flux of gas across the membrane. In some approaches, a thickness greater than 500 μm may be preferable in order to gain mechanical strength.

In various approaches, the flux into a membrane (e.g., film, wall, etc.) may be determined by the material of the membrane. For example, if the material is reactive, the flux into the membrane may be slowed, lower, higher, etc. In some approaches, the flux may be independent of the thickness. For example, a membrane loaded with live whole cells could deplete the methane before it diffuses across the membrane, such that the center of the membrane may not contribute to reactivity (e.g., methane consumption). In this case, increasing the thickness of the membrane only increases the unproductive center region and does not change the flux.

Example of a Bioreactor to Convert Methane to Methanol

The only known true catalyst (industrial or biological) to convert methane to methanol under ambient conditions with 100% selectivity is the enzyme methane monooxygenase (MMO), found in methanotrophic bacteria, which converts methane to methanol according to the following reaction in Equation 2:

$${CH}_{4} + {O}_{2} + 2 {e}^{-} + 2 {H}^{+} {\to }^{ pMMO } {CH}_{3} OH + {H}_{2} O \qquad Equation 2$$

Partial methane oxidation by MMO enzymes can be carried out using whole methanotroph organisms, but this approach inevitably depends on energy for upkeep and metabolism of the organisms, which reduces conversion efficiency. Moreover, biocatalysis using whole organisms is typically carried out in low-throughput unit operations, such as a stirred-tank reactor.

One industrial-biological approach may therefore include separating the MMO enzyme from the host organism. Isolated enzymes may offer the promise of highly controlled reactions at ambient conditions with higher conversion efficiency and greater flexibility of reactor and process design. MMOs have been identified in both soluble MMO (sMMO) and particulate (pMMO) form. The use of pMMO has advantages for industrial applications because pMMO comprises an estimated 80% of the proteins in the cell membrane. Moreover, isolating the membrane fraction of the lysed cells by centrifugation provides a reasonably pure concentrated pMMO.

Traditional methods of enzyme immobilization and exposure to reactants are not sufficient to use pMMO effectively. These typical methods include cross-linking enzymes or immobilizing them on a solid support so that they can be separated from the products and carrying out batch reactions in the aqueous phase in a stirred-tank reactor. As discussed previously, operation of a stirred-tank reactor has several drawbacks, including low productivity, high operating costs, loss of catalytic activity due to enzyme inactivation, and variability in the quality of the product. The stirred-tank reactor is also not the optimal design for gas to liquid reactions such as methane to methanol conversion, as it does not allow efficient delivery of reactant gases to enzymes or organisms in the bulk solution. Gas delivery in stirred-tank reactors is often achieved by bubbling the gas through the liquid, but this approach suffers from mass-transfer limitations. Furthermore, methane and oxygen are only sparingly soluble in aqueous solvents: 1.5 mM/atm and 1.3 mM/atm respectively at 25° C. Reactant concentrations are necessarily solubility-limited when the enzymes or organisms are dispersed in the aqueous phase.

Moreover, another reason as to why the pMMO enzyme is not amenable to standard immobilization techniques designed for soluble proteins is due to the fact that surfactant solubilization of isolated pMMO leads to a pronounced reduction in activity. For example, high surface area porous inorganic supports have been extensively studied and implemented for immobilizing soluble enzymes and have been shown to enhance enzyme stability while achieving high enzyme loading in nanometer scale pores. The majority of the surface area in mesoporous materials is accessible only to proteins significantly smaller than 50 nm and would therefore be inaccessible to the large (>100 nm), optically opaque vesicles and liposomes that comprise pMMO in crude membrane preparations.

Accordingly, the exemplary aspects discussed in therein are directed toward advances in biocatalytic processes, e.g., for selective methane conversion. For instance, some exemplary aspects are directed toward a biocatalytic material comprising pMMO and/or whole cells embedded in polyethylene glycol diacrylate (PEGDA) hydrogel. Embedding enzymes, such as pMMO, and/or whole cells that operate on gas phase reactants within the solid, gas permeable polymer hydrogel allows tuning of the gas solubility, permeability, and surface area thereof. An additional advantage to immobilizing pMMO and/or whole cells within the polymer hydrogel, rather than on the surface of an impermeable support, is the potential to fully embed pMMO and/or whole cells throughout the depth of the polymer hydrogel for high loading.

In some approaches, an acrylate-functionalized PEG (e.g., PEGDA, PEGTA, etc.) may be selected as a primary polymer substrate because of its biocompatibility and flexibility for further development. The acrylate-functionalized PEG may be physically or chemically combined with hydrophobic polymers in additional approaches for enhanced gas solubility and transport in various approaches. Moreover, the pMMO and/or whole cells embedded acrylate-functionalized PEG hydrogel may be amenable to various forms of 3D-printing, which offers the ability to rapidly prototype structures, tune micron to centimeter-scale material architecture, and precisely tailor structures for the system configuration and mass transfer, heat, and diffusion limitations.

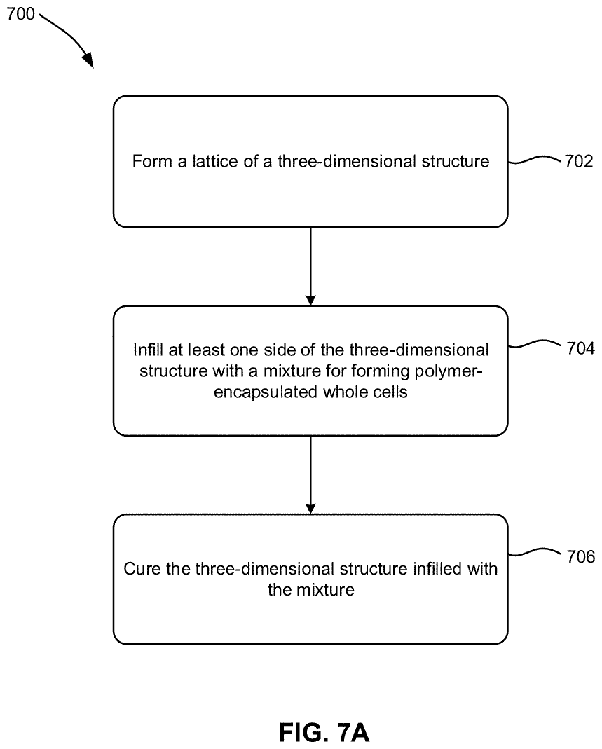

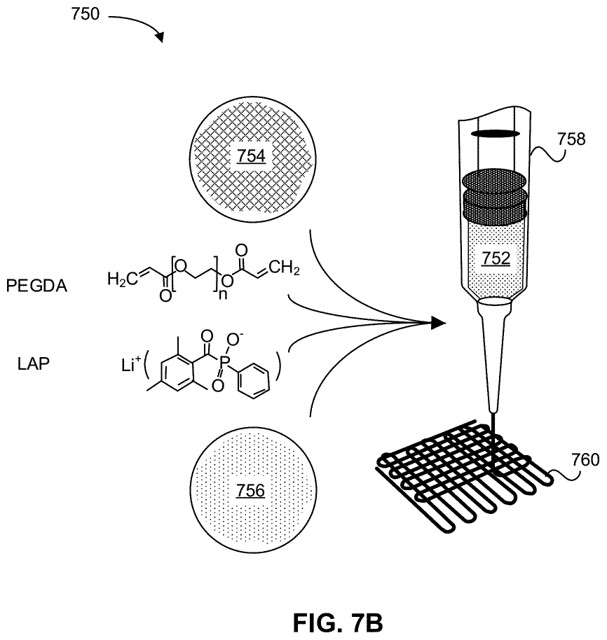

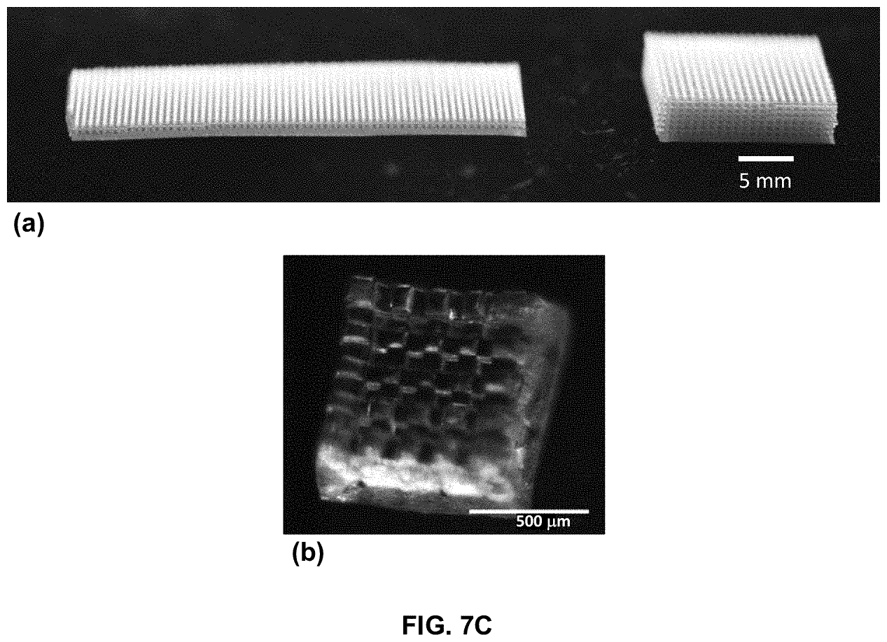

Referring now to FIG. 7A, an exemplary method 700 of forming a bioreactor (such as those disclosed herein) is shown, according to one inventive concept. As an option, the present method 700 may be implemented in conjunction with features from any other inventive concept listed herein, such as those described with reference to the other FIGS. Of course, the method 700 and others presented herein may be used in various applications and/or in permutations, which may or may not be specifically described in the illustrative inventive concept listed herein. Moreover, more or less operations than those shown in FIG. 7A may be included in method 700, according to various inventive concept. Furthermore, while exemplary processing techniques are presented with respect to FIG. 7A, other known processing techniques may be used for various steps.

As shown in FIG. 7A, the method 700 includes forming a lattice of a 3D structure using an additive manufacturing technique. In some approaches, the lattice may be formed via projection microstereolithography (PμSL) or extrusion-based printing, (e.g., direct ink writing). A 3D structure is defined as a structure having three dimensions: a length, a width, and a height. In one approach, the 3D structure may be a film having a plurality of layers, where the film has a thickness (e.g., a height, a depth, etc.) of greater than about 10 μm, a width, and a length. In one approach the 3D structure is a film having the geometry of a lattice structure.

In some approaches, a thickness of the at least one side (wall, sidewall, edge, etc.) of the 3D structure is in a range of about 10 μm to about 5000 μm (5 mm). In preferred approaches, a thickness of at least one side of the 3D structure is in a range of 10 μm to about 500 μm.

As discussed above, a printed 3D structure may be in the form of a tube having a wall. In some approaches, operation 704 of method 700 includes infilling at least one side (e.g., wall, sidewall, border, edge, etc.) of the printed 3D structure with a mixture for forming polymer-encapsulated whole cells. In some approaches, the preferred thickness of the 3D structure provides the preferred optimal density of whole encapsulated cells. For example, in one approach, a thickness of the 3D structure includes whole encapsulated cells having an OD of 20.

In various approaches, a concentration of whole cells in the mixture of polymer-encapsulated whole cells has a cell optical density in a range of about 4.0 to about 160. In preferred approaches, a concentration of whole cells in the mixture of polymer-encapsulated whole cells has a cell optical density in a range of about 10 to about 80.

Operation 706 of method 700 includes curing the 3D structure infilled with the mixture. In one approach, operation 706 includes curing a printed 3D structure infilled with the mixture. In some approaches, the curing may include UV radiation for an effective amount of time to cross-link the polymer in the mixture such that the whole cells are encapsulated in the polymer. In some approaches, the duration of curing by UV radiation may convert greater than 50% of the pre-polymer to crosslinked polymer. In some approaches, the duration of curing of the mixture by UV radiation may be up to 5 minutes. In preferred approaches, the duration of curing by UV radiation may be under one minute. In exemplary approaches, the duration of curing by UV radiation may be in a range of 10 seconds to 30 seconds.

On one concept, the polymer-encapsulated whole cells may be used as an ink to form a printed 3D structure. In some approaches, an additively manufactured reactor may operate with high cell densities that is typically not feasible with a conventional stirred-tank reactor. Thus, an additively manufactured reactor may be a major contributor to process intensification. FIG. 7B is a schematic drawing of a process 750 including a DIW apparatus 758 with novel ink 752 formulations comprised of nanocellulose crystals 754, an acrylate-functionalized PEG such as PEGDA, photoinitiator LAP, and yeast 756, according to one inventive concept. In some approaches, the yeast 756 may include Saccharomyces cerevisiae (S. cerevisiae). Adjusting the PEGDA polymer-cell formulation with nanocellulose crystals 754 or dry yeast 756 enables a DIW ink 752 that is photo-curable and may be used to directly print lattices 760 of cells encapsulated in PEG, as shown in FIG. 7B, according to one aspect. Using this approach, the inventors have demonstrated an ink formulation and DIW technique with polymer-cell formulations containing live yeast as a model for bacterial cells before incorporating methanotrophs.

In some approaches, the polymeric network may also include enzymatic reactive components that may comprise any of the enzymatic reactive components disclosed herein including, but not limited to, isolated enzymes, trans-cell-membrane enzymes, cell-membrane-bound enzymes, liposomes coupled to/comprising an enzyme, combinations thereof, etc. Moreover, as discussed previously, the enzymatic reactive components may be embedded/incorporated into the polymeric network via several methods including, but not limited to: attaching the enzymatic reactive components to electrospun fibers of a first polymer, and backfilling with a second polymer (see, e.g., the method 400 described in FIG. 4); directly incorporating the enzymatic reactive component into a polymer or block-copolymer network before or after crosslinking the network (see, e.g., the method described in FIG. 5); and other suitable incorporation methods as would become apparent to one having skill in the art upon reading the present disclosure.

The polymeric network may include any of the materials, and/or be of the same form, as any of the polymeric networks disclosed herein. For instance, this polymer network may be configured to serve as a mechanical support for the enzymatic reactive components embedded therein, as well as include nanometer scale domains of higher permeability to the first fluid and nanometer scale domains of higher permeability to the second fluid. Moreover, in some approaches, the polymeric network may include at least a two phase polymer network, e.g. a polymer network comprising two or more polymeric materials. In other approaches, the polymeric network may include a mixture of at least one polymer material and at least one inorganic material.

As indicated above, the polymeric network may be configured to separate a first and second fluid associated with a reaction catalyzed by the enzymatic reactive components embedded therein. The first and second fluids may be two different fluids, such as liquids and gasses, an aqueous species and a non-aqueous species, a polar species and a non-polar species, etc.

As also shown in FIG. 7B, the process 750 includes fabricating and patterning one or more layers in the membrane material via a 3D printing process. See also operation 704 of FIG. 7A. In preferred approaches, the 3D printing process includes a projection microstereolithography (PμSL) process as known in the art. In various approaches, each layer in the membrane material patterned/formed via the desired 3D printing process may include a plurality of 3D structures (e.g., hollow fibers, micro-capsules, hollow tube lattices, spiral wound sheets, etc.) configured to optimize the bioreactor geometry (and surface area) for mass transfer, reaction rate, product removal, continuous processing, etc. Photographs of several exemplary PEG-pMMO 3D structures formed/patterned according to a PμSL process are shown in FIG. 7C.

As discussed in greater detail below, the novel bioreactors described herein, such as described in FIG. 6A, may be particularly configured for methane activation with an energy efficiency from greater than or at least equal to about 68%. In such an approach, the enzymatic reactive components embedded within the polymeric network may include pMMO to covert methane reactants, CH₄, to methanol products, CH₃OH. Preferably, this engineered pMMO may exhibit a specific activity greater than about 5 μm/(g·s) and/or a turnover frequency greater than about 10/s. Additionally, the amount of the engineered pMMO in such bioreactors may be about 50 g per L of reactor volume.

In some approaches, a reducing agent may be included with the aforementioned engineered pMMO to assist in methane conversion. However, in other approaches, the engineered pMMO may not need such a reducing agent or be configured to accept electrons via direct electron transfer. For instance, as shown in Table 2, the methane conversion may proceed by: (1) using pMMO configured to use methane as a reducing agent (Reaction 1); (2) supplying electrons directly to the pMMO (Reaction 2);

TABLE 2
Reactions Pathways of Methane Conversion

| | | Energy | Carbon |
|---|---|---|---|
| | Reaction Pathway | Efficiency | Efficiency |
| Reaction 1 | 2CH₄ + O₂ → 2CH₃OH | 80% | 100% |
| Reaction 2 | CH₄ + O₂ + 2H⁺ + 2e⁻ → | >65% | 100% |
| | CH₃OH + H₂O | | |
| Reaction 3 | 4CH₄ + 3O₂ → 3CH₃OH + CO + | 68% | 75% |
| | 2H₂O | | |

and (3) using H₂ gas. Yet another reaction pathway may involve steam reformation as shown in Reaction 3.

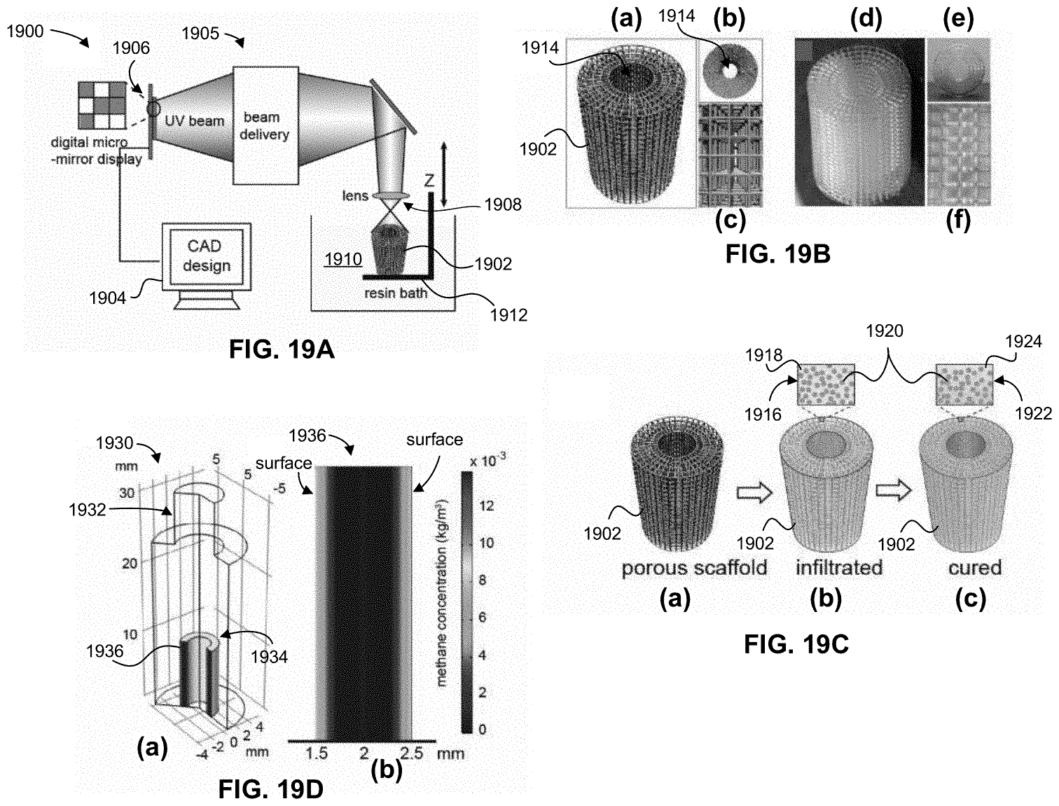

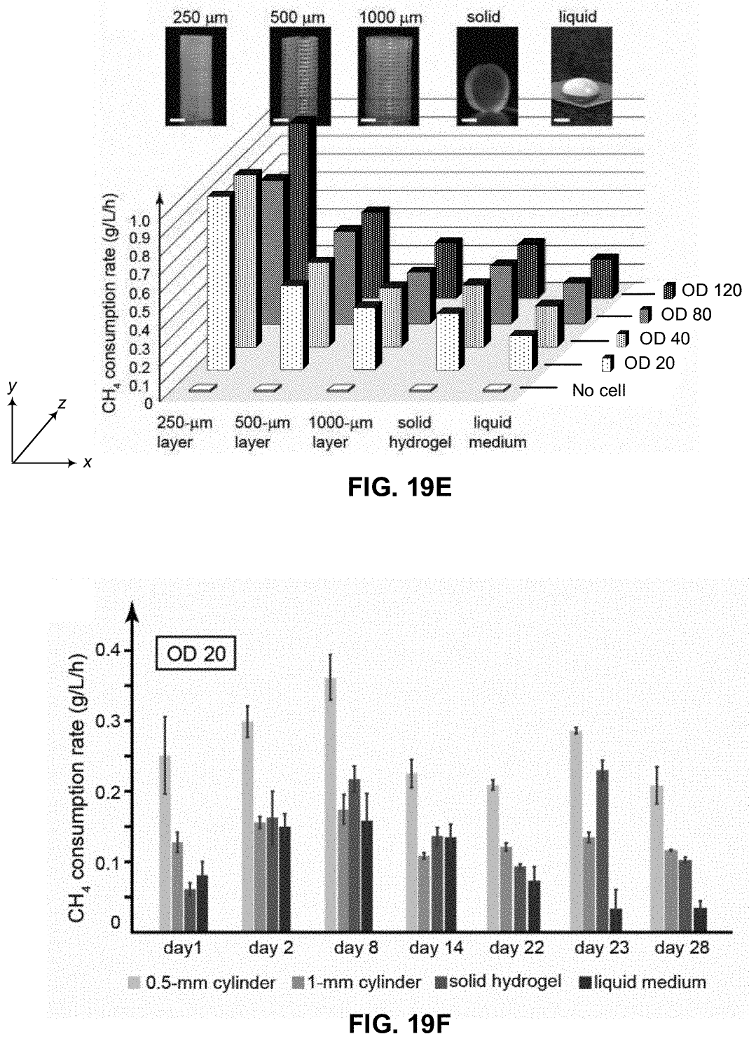

FIG. 19A is a schematic drawing that describes the process 1900 of forming a scaffold 1902 for a bioreactor, according to one embodiment. In one approach, the process 1900 describes operation 702 of method 700 (see FIG. 7A). As shown in FIG. 19A, a computer-aided design (CAD) of a scaffold (e.g., lattice, geometric 3D structure, etc. as illustrated in part (a) of FIG. 19B) is created on a computer 1904. As shown in FIG. 19A, a UV projection system 1905 forms the scaffold 1902 for the bioreactor (e.g., as shown in as a bioreactor 1520 in part (a) of FIG. 15B). Briefly, an image 1906 is projected as a pattern 1908 into a vat 1910 of resin that solidifies as the projected pattern 1908 on a substrate 1912. The substrate 1912 moved down in a z-direction as subsequent layers of the projected pattern 1908 are added to the scaffold 1902. The result of the process 1900 is a formed 3D scaffold 1902 as shown in the image of part (d) of FIG. 19B.

Parts (a) through (c) of FIG. 19B illustrate different views of a CAD of a scaffold to be formed. Part (a) is a perspective view of the scaffold 1902 that shows the hollow center 1914 of the scaffold and the lattice pattern. Part (b) is a top view down the axis of the scaffold 1902 and the hollow center 1914. Part (c) is a magnified view of the lattice pattern of the scaffold.

Parts (d) through (f) of FIG. 19B illustrate different views of the formed 3D scaffold following the process as described in FIG. 19A. Part (d) is a perspective view of the formed scaffold. Part (e) is a top view down the axis of the scaffold and the hollow center of the formed cylinder-shaped 3D structure. Part (f) is a magnified view of the lattice pattern of the formed scaffold.

In one approach, the schematic drawings of FIG. 19C illustrate operation 704 of method 700 (see FIG. 7A) of infilling a 3D structure with a mixture for forming polymer-encapsulated whole cells. Part (a) of FIG. 19C shows the porous scaffold formed by process 1900 in FIG. 19A. In one approach, the porous structure may be treated with oxygen to enhance the hydrophilicity of the structure.

In Part (b) the porous scaffold is infiltrated (e.g., infilled, soaked, etc.) with a mixture 1916 of hydrogel 1918 and encapsulated whole cells 1920 (as shown in inset). The mixture 1916 may infiltrate the pores of the scaffold 1902 by capillary force. Part (c) describes the curing step, as described in one approach for operation 704 of method 700 (see FIG. 7A), where the scaffold 1902 with infiltrated mixture 1916 of hydrogel 1918 and encapsulated whole cells 1920 is cured into a cured mixture 1922 that locks the cells 1920 in place in the cured hydrogel 1924 within the lattice pattern of the sidewalls of the scaffold 1902.

FIG. 19D describes the computational simulation of the methane concentration inside the structure where the methane gas surrounding the apparatus 1930 and hydrogel cylinder 1934 is static without gas flow, and thus gas absorption is diffusion based. Part (a) shows the wire frame 1932 that is a 5/8 section cut of an apparatus 1930 being run. The hydrogel cylinder 1934 is the light and dark shaded cylinder structure (5/8 section) with a vertical cross-section of the sidewall 1936 of the hydrogel cylinder 1934 exposed. Part (b) shows the methane concentration profile of the vertical cross-section of the sidewall 1936 from the drawing in part (a). The scale of shading to methane concentration (kg/m³) is shown in the vertical bar on the right of part (b). According to the shading scale, the vertical cross-section of the sidewall 1936 demonstrates a high methane concentration on the surface (light shading) that quickly depletes (to a darker shading) toward the center of the sidewall 1936. Thus, the center region of the cross-section of the sidewall 1936 may be a dead volume section where there is no methane. Without wishing to be bound by any theory, it is believed that the methane has been consumed by the whole cells in the cured hydrogel of the sidewall of the cylinder close to the service, and thus there is no methane available for consumption in the center of the sidewall. Further, in one approach as shown in part (b) of FIG. 19D, the methane is absorbed at each surface on opposite sides of the sidewall.

Methane consumption may be determined by the geometry of the 3D structure infiltrated with cured hydrogel and whole encapsulated cells. FIG. 19E shows a plot of different geometries of 3D structures along the x-axis. These include a hydrogel cylinder 250 μm sidewall, a hydrogel cylinder with a 500 μm sidewall, a hydrogel cylinder with a 1000 μm, a solid hydrogel disc, and liquid medium. According to these results, and these are example only and not meant to be limiting in any way, methane consumption (along the y-axis) is most efficient with the hydrogel cylinder having a 250 μm sidewall compared to the other geometries, thereby indicating that a thicker sidewall may not indicate improved methane consumption.

In one example, the plot of FIG. 19E demonstrates that the optical density (OD) of the whole cell in the hydrogel infiltrations with structures of different geometries does not show remarkable differences at the concentrations of optical density (along the z-axis) tested in these geometries. At OD of 20 through OD of 120, the cells showed comparable methane consumption depending on the geometry of the 3D structure. These results are by way of example only and are not meant to be limiting in any way.

In various embodiments, 3D structures infiltrated with cured hydrogel and encapsulated whole cells show sustained methane consumption for more than three weeks, as shown in the plot depicted in FIG. 19F. In one approach, a hydrogel/cell cylinder with sidewalls having a thickness of 500 μm perform show a moderate decrease in methane consumption at two weeks, and then sustains the level of methane consumption for a further two weeks. A thicker hydrogel cylinder (1 mm sidewall), solid hydrogel disc, and liquid suspension of cells all showed comparable methane consumption for first two weeks, but the liquid suspension of cells demonstrated a notable drop in methane consumption for the following two weeks (week 3 and week 4). In various approaches, the 3D structures infiltrated with cured hydrogel/whole cells demonstrate longevity of functional processes, e.g., methane consumption, for a duration of nearly a month.

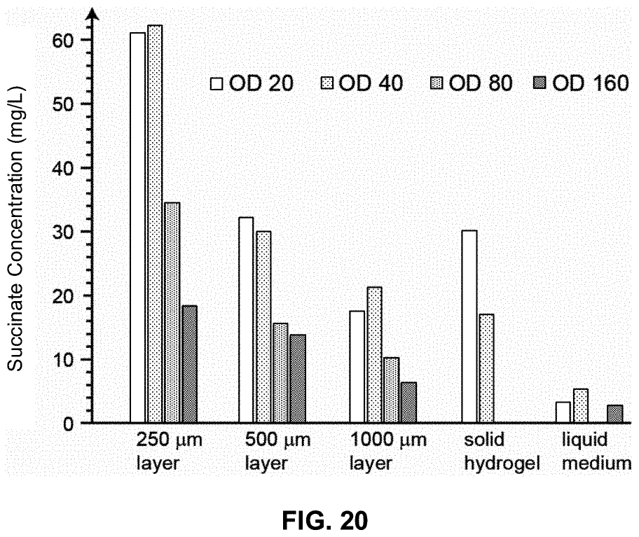

In various approaches, one of the products of methane consumption may include the production of succinate. In one approach, 3D structures infiltrated with cured hydrogel/whole cells produce the organic acid succinate, as shown in the plot of FIG. 20. In one approach, the production of succinate via methane consumption in 3D structures infiltrated with hydrogel and whole cells may be determined by the geometry of the 3D structure. In one approach, higher concentrations of succinate were produced in hydrogel cylinders compared to the solid hydrogel having whole cells or liquid suspension of cells.

In one approach, the production of succinate may be determined from the optical density of the whole cells. In one approach, a hydrogel cylinder having a sidewall thickness of 250 μm and infiltrated with whole cells at an OD of 20 to OD of 40 produce significant levels of succinate, greater than 50 mg/mL. In some approaches, increasing the concentration of whole cells to an optical density of 80 and 160 in the hydrogel of the 3D structures may have a less than optimal effect on succinate production. These results are by way of example only and are not meant to be limiting in any way.

Experiments/Examples

The following experiments and examples pertain to various non-limiting aspects of the bioreactors described herein. In particular, the following experiments and examples are directed to bioreactors comprising pMMO embedded in a polymeric network for the conversion of methane to methanol. It is important to note that the following experiments and examples are for illustrative purposes only and do not limit the invention in anyway. It should also be understood that variations and modifications of these experiments and examples may be made by those skilled in the art without departing from the spirit and scope of the invention.

Experiment Results

Encapsulation of Whole Cells in PEG Hydrogel

In one aspect, whole M. capsulatus Bath and M. buryatense cells were encapsulated in various polymers and/or biomaterials including PEGDA, gelatin, and cellulose nanocrystals. The polymer concentration may be varied from 10-50% polymer by weight depending on the type of pre-polymer used and the cell optical density (OD) may be varied in a range from 4 to 80. In one aspect, PEGDA with molecular weights ranging from 575-20,000 Da was employed. The cells were mixed with the pre-polymer formulations and the photoinitiator lithium phenyl-2,4,6-trimethylbenzoylphosphinate (LAP) was added prior to curing at 405 nm for 10 seconds.

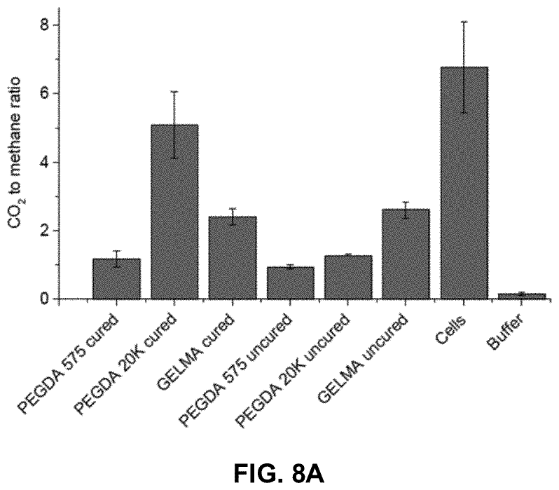

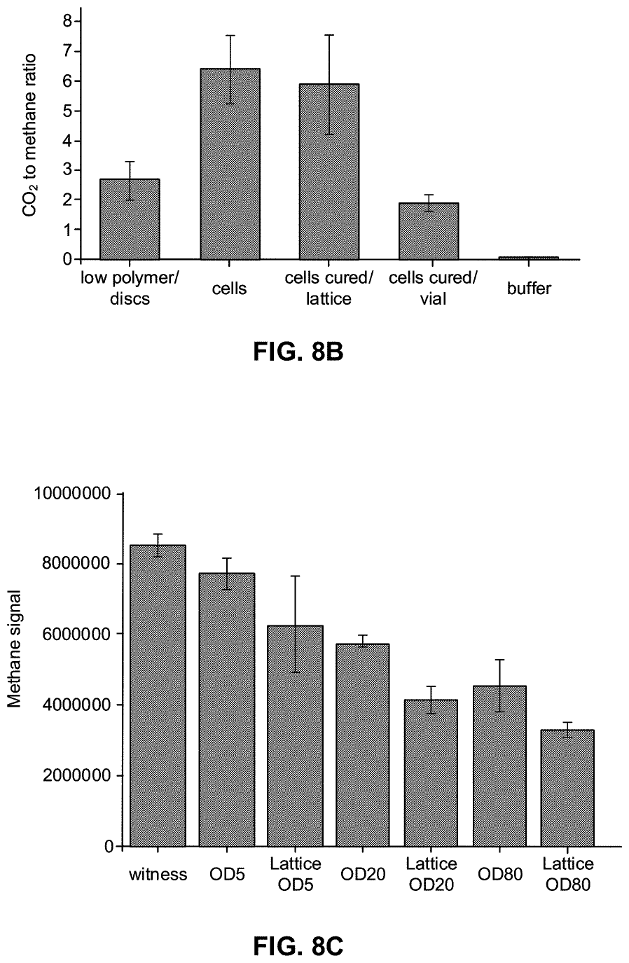

A particularly preferred formulation includes using M. capsulatus Bath cells in an amount corresponding to about OD 40 and 12 wt. % PEGDA (MW=20 kDa). The formulation may be cured for 10 seconds and activity is shown by the CO₂ (product) to methane (reactant) ratio in FIG. 8A.

The polymer-cell formulation described herein may be cured within structure lattices that were made with PμSL or DIW technology. The lattices were, in one approach, composed of a silicone polymer, and the geometry and lattice structure easily modified. According to one aspect, a lattice was created with PμSL (as shown in FIG. 6B part (a)). The lattice was designed to be a tube structure with the walls infilled with the polymer-cell solution and then cured at 405 nm with the center of the tube remaining hollow, to be filled with buffer during the catalytic reaction. The lattice as shown in part (a) may be suitable for use with methanotroph cells. In another aspect, a lattice mesh was created with DIW (as shown in FIG. 6B part (b)). The silicone structure as shown in part (b) may be suitable for use with methanotroph cells.

FIG. 8B is a plot the ratio of CO₂ (product) to methane (reactant) of methanotroph cells in various geometries and structures. By testing the polymer formulation in different geometries, the inventors found that the polymer-cell formulation cured within the PμSL printed lattice tube performs better than expected, e.g., as well as the control experiment as shown in FIG. 8B.

FIG. 8C is a plot of methane consumption of methanotroph cells at varying cell densities in solution compared to varying cell densities in lattice structures. These results how that it is possible to vary the cell density of the cells encapsulated within the lattice and achieve an optical density in a range from about OD5 to about OD80, as shown in FIG. 8C.

pMMO Activity in PEG Hydrogel

Several methods for embedding pMMO in a PEGDA based polymer hydrogel were explored to enable its use as a biocatalytic material which could be molded into controlled, predetermined structures with tunable permeability and surface area for practical use. Initial efforts focused on solubilizing the crude membrane preparations using surfactant so that the material could be incorporated homogeneously in the polymer. It was discovered that any contact of the crude membrane preparations with surfactant, including encapsulation in nanolipoprotein particles, led to a pronounced decrease in activity. However, mixing the crude membrane fractions, either as prepared or extruded as liposomes directly with low concentrations of PEGDA 575 gave promising results. According the experiments described in this section focused on optimizing the activity and protein retention of crude membrane preparations with PEGDA 575.

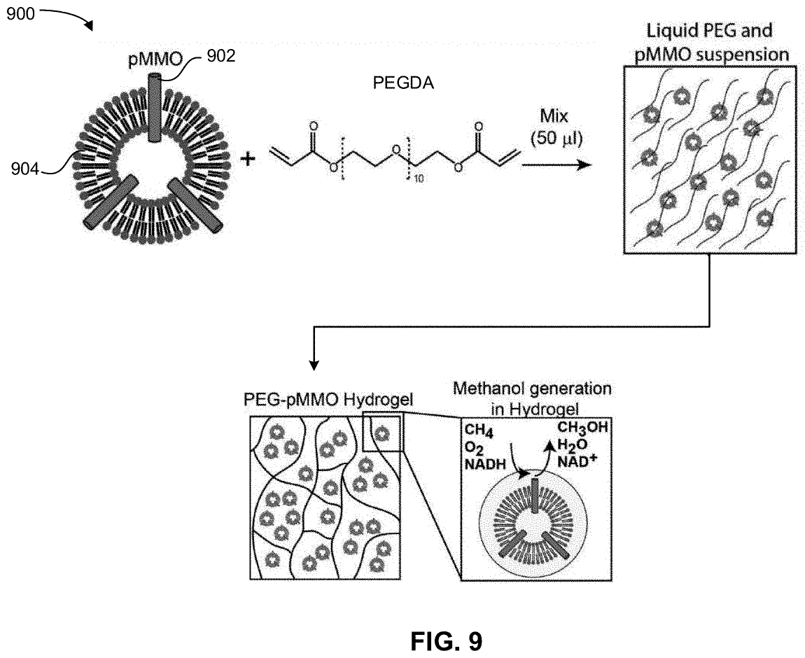

A schematic of the method 900 used to fabricate the PEG-pMMO hydrogels is shown in FIG. 9. The synthesis of the PEG-pMMO materials includes only membrane 904, membrane bound pMMO 902, PEGDA macromer, photoinitiator (not shown), and ultraviolet (UV) light. Photoinitiator concentrations higher than 0.5 vol % in PEGDA decreased the pMMO activity, therefore the photoinitiator concentration was held constant at 0.5 vol %.

Membrane bound pMMO alone in each activity assay was used a positive control. The measured activity of the membrane bound pMMO alone was highly variable from experiment to experiment, from about 75 to 200 nmol MeOH mg⁻¹ min⁻¹, while the optimized PEG-pMMO samples were less variable, in a range from 65 to 128 nmol MeOH mg⁻¹ min⁻¹. The measured activity for both membrane bound pMMO alone and immobilized pMMO were similar to known values for membrane bound pMMO with methane as a substrate: 25-130 nmol MeOH mg⁻¹ min⁻¹.

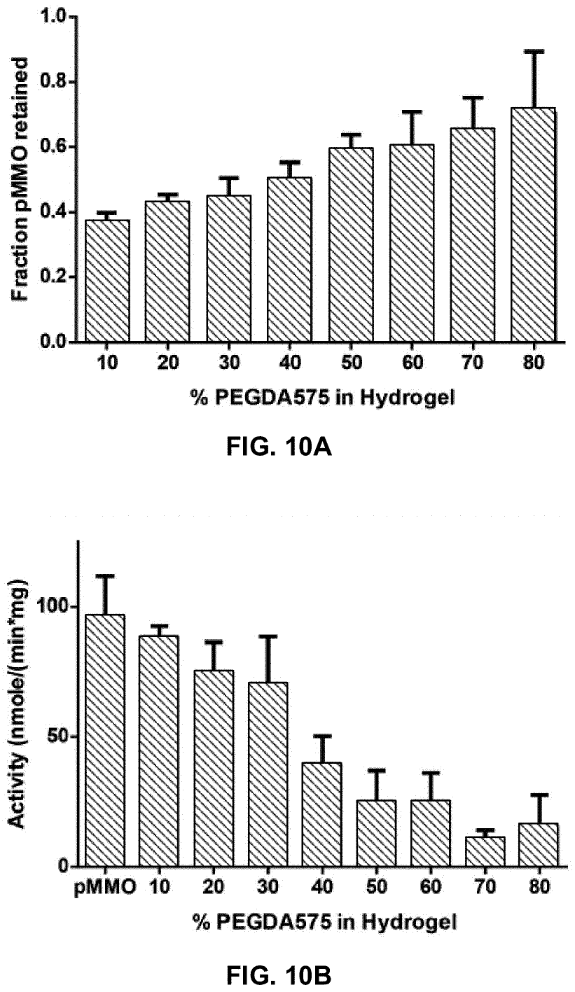

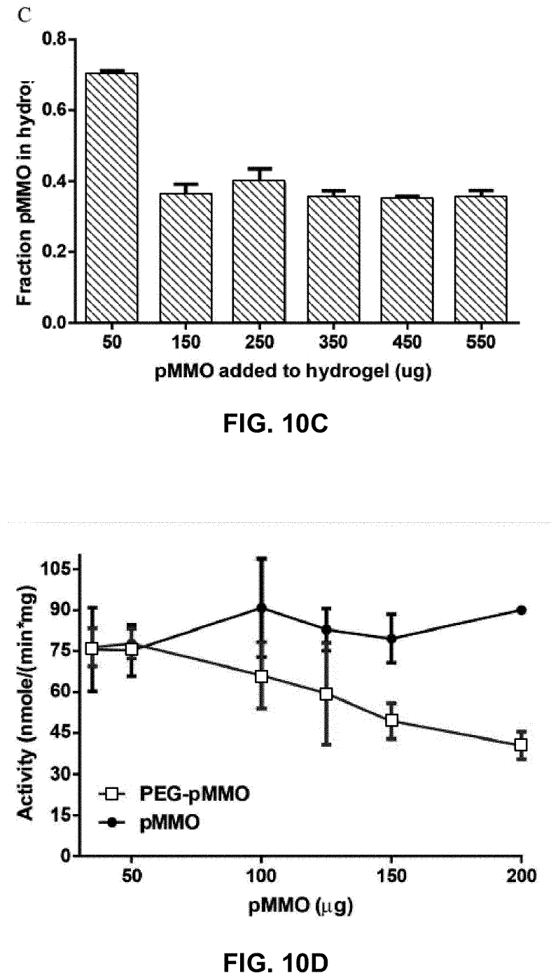

FIGS. 10A-10D shows the results from systematically increasing the volume % of PEGDA in the solution prior to curing on protein retention (FIGS. 10A, 10C) and activity (FIGS. 10B, 10D). Mixing the pMMO solution with PEGDA at the appropriate vol % (10-80%), and UV curing resulted in 50 μl solid PEG-pMMO hydrogels. As the PEGDA vol % was increased from 10-80%, the overall stiffness of the material increased and the amount of residual liquid on the surface of the hydrogel decreased. A gradual increase was observed in the fraction of pMMO that was retained (0.4-0.75) when the PEGDA vol % was increased from 10-80% (FIG. 10A).

However, a dramatic decrease in pMMO activity was observed as the PEGDA vol % was increased (FIG. 10B). At 10% PEGDA, the pMMO activity was approximately 88+/−4 nmol MeOH min⁻¹ mg⁻¹, which closely corresponded to the activity of pMMO alone (96+/−15 nmol MeOH min⁻¹ mg⁻¹) (FIG. 10B). This value dropped below 30 nmol MeOH min⁻¹ mg⁻¹ when the PEGDA vol % was greater than 50% (FIG. 10B). The amount of pMMO retained in the hydrogel before and after the activity assay did not change, indicating that no pMMO leached out during the activity assay and the enzyme was efficiently entrapped in the hydrogel. These combined findings suggest that one considers both pMMO retention and activity when identifying the optimal PEGDA vol %. Since only a marginal increase in pMMO retention (0.4 vs 0.42) and a more significant decrease in pMMO activity (88 vs 74 nmol MeOH min⁻¹ mg⁻¹) was observed when the PEGDA vol % was increased from 10% to 20%, all remaining experiments were performed using 10 vol % PEGDA.

FIGS. 10C and 10D illustrate the effect of varying the concentration of pMMO during hydrogel fabrication on pMMO retention and activity. For these experiments, the amount of pMMO used to generate the 50 μl PEG-pMMO hydrogel was varied between 50 μg and 550 μg. The fraction of pMMO retained was the highest at the lowest pMMO concentration tested (50 μg −0.75 retained) and a dramatic decrease was observed when the pMMO was increased to 150 μg (−0.4 retained) (FIG. 10A). Further changes in the total pMMO retained was not observed when the pMMO was increased up to 550 μg. To assess the effect of varying the pMMO concentrations in the PEG-pMMO hydrogel on activity, PEG-pMMO hydrogels were prepared with 50-550 μg of pMMO, which resulted in retention of 35-200 μg of pMMO in the hydrogel, and the activity was measured. As shown in FIG. 10B, pMMO activity in the hydrogel was similar to the activity of pMMO alone when the amount of pMMO retained was below 50 μg; however, there was a gradual decrease in pMMO activity in the hydrogels as the pMMO levels were increased from 50-200 μg, which was not observed in the pMMO alone sample (FIG. 10D).

Preserving the native activity of pMMO in the PEG hydrogel includes a balance between pMMO loading and enzyme activity. Higher polymer concentrations gave rise to higher pMMO loading and retention (FIG. 10A). Increasing the polymer concentration also correlated with diminished pMMO activity. This trend may be due to reduced polymer permeability or enzyme degradation by acrylate groups and/or free radicals at higher polymer concentrations. While it has been shown that PEDGA concentration (and by correlation, crosslinking density) has minimal effect on methane permeability in the gas phase, gas permeability is affected by the hydration (swelling) of hydrogel materials. Thus, PEGDA concentration may impact methane permeability in swollen PEG-pMMO. Higher PEGDA concentrations also decrease the distance between crosslinks and the diffusion of aqueous solutes through the hydrogel. Therefore, higher PEGDA concentrations may limit diffusion of the NADH cofactor to the enzyme or diffusion of the methanol product from the active site. Additionally, photo-initiated cross-linking reaction used to generate the cross-linked hydrogel results in the generation of free radicals, which can result in the oxidation of amino acids in proteins and cleavage of peptide bonds. The optimized PEG-pMMO formulations described in the text were remarkable in that they preserved physiological pMMO activity in a polymeric material. For approaches including a higher protein or polymer content, enzyme degradation and free radicals may be managed by changing the macromer length and/or curing chemistry, thereby increasing hydrogel mesh size (promoting diffusion) and reducing the number of radicals generated.

Reuse and Stability of PEG-pMMO Hydrogels

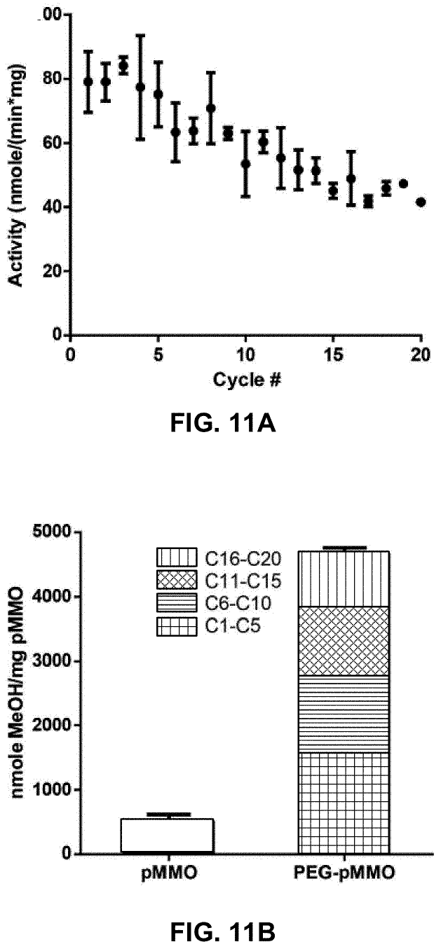

The development of fully active pMMO in a polymer material allowed the reuse of pMMO without painstaking centrifugation with each new set of reactants. Measurements were made regarding the effects of reuse of the PEG-pMMO hydrogel on overall enzyme activity and methanol generation using PEG-pMMO that was prepared with an initial pMMO amount of 150 μg and 10 vol % pMMO (FIGS. 11A-11B). In these experiments, the PEG-pMMO hydrogels were subjected to 20 cycles of 4 min exposures to methane. The hydrogel was washed thoroughly between each cycle to ensure that no residual methanol product remained in the hydrogel between cycles. The protein content in the reaction buffer for each cycle was measured to verify that the pMMO concentrations remained constant, and that there was no leaching through the course of the study. As shown in FIG. 11A, the activity between assay cycles 1 to 5 remained close to the initial activity (˜80 nmol MeOH min⁻¹ mg⁻¹) and then gradually decreased to ˜45 nmol MeOH min⁻¹ mg⁻¹ after 20 cycles. The error bars correspond to the standard deviation from the average of four replicates. FIG. 11B shows the cumulative methanol produced from these 20 consecutive reactions of PEG-pMMO compared to a single reaction of membrane bound pMMO. Immobilization of fully active pMMO in a material allowed the facile production of 10 fold more methanol per protein than could be produced with membrane bound pMMO (which can only be reused with painstaking repeated centrifugation and rinsing steps).

Continuous Flow-Through Bioreactor

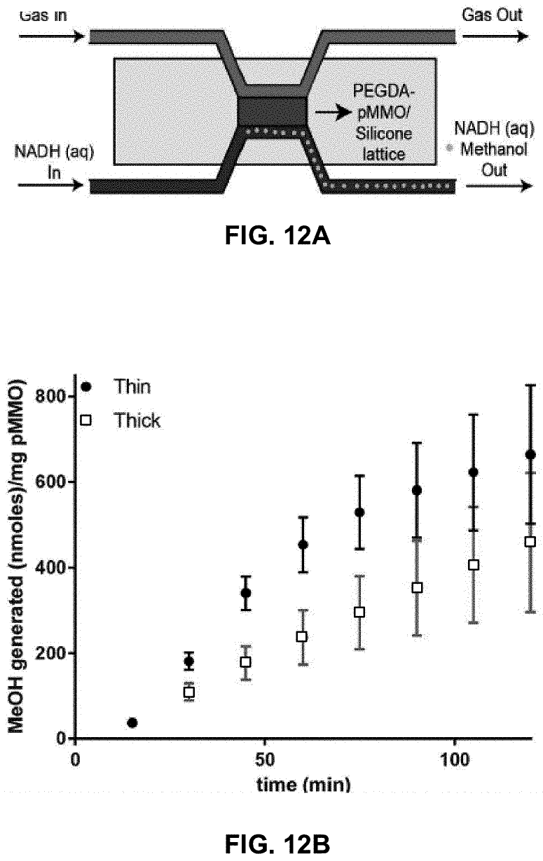

Establishing that that the PEG-pMMO material could be reused with no measurable protein leaching indicated that the material would be amenable for use in a bench-scale continuous flow reactor. A design where the pMMO material is suspended between gas and liquid reservoirs was discovered herein as desirable given that pMMO acts upon gas phase reactants and generates liquid phase. However, PEG-pMMO, and hydrogels in general, are mechanically brittle and difficult to handle when molded as thin membranes. Accordingly, the PEG-pMMO material was embedded into a 3D silicone lattice (printed using Direct Ink Write) in order to greatly increase the mechanical stability and to easily tune the size and shape of the hydrogel for use in a continuous reactor (FIG. 12A). As discussed in greater detail below, the lattice was constructed of 250 micron silicone struts and contained 250 micron void spaces (50% porosity) which were then infilled with PEGDA 575, crude pMMO membrane preparations, and photoinitiator and crosslinked in place with ultraviolet light. Two such lattice structures, thin and thick, were designed to compare effects of PEG-pMMO surface area to volume ratio on methanol production. The surface area to volume ratio of thin vs. thick for these experiments was 5 to 1. The silicone lattice structure increases the bulk gas permeability of the materials, since silicone permeability is at least 50 times greater than the PEGDA hydrogel permeability.

The resulting hybrid silicone-PEG-pMMO lattice materials were mechanically robust, allowing the suspension of the PEG-pMMO lattice of 1 millimeter thickness between gas and liquid reservoirs in a flow-through reactor. A schematic of the reactor cross section is shown in FIG. 12A. With this configuration, a methane/air gas mixture was flowed on one side of the lattice and the NADH was introduced on the other side, while continuously removing and collecting methanol in buffer. In order to determine the length of time the membrane could be continuously used, the cumulative methanol produced per mg of enzyme was measured at 25° C. at 30 min intervals in the thick lattice over the course of 5.5 hours. The methanol production rate (slope of methanol vs. time curve) was stable for about 2.5 hours and declined gradually over the next 3 hours. In order to evaluate whether the geometry of PEG-pMMO material influenced methanol production rates, reactor outlet fractions from reactors containing the thin and thick lattices were compared at 15 min intervals at 45° C. over the course of two hours (FIG. 12B) in triplicate. The methanol concentrations produced in the flow reactor were on average 12 and 6% of what was predicted, for thin and thick lattices, respectively, based upon analyte flow rates and an assumed pMMO activity of 80 nmol MeOH min⁻¹ mg⁻¹. The low concentration values relative to predicted values may be due to lower actual pMMO concentrations in the material than was calculated. As shown in FIG. 12B, the methanol produced (per mg of protein) by the thin membrane was double that produced by the thick membrane over the course of the first hour. Over the following hour, the methanol production rate by the thin membrane declined relative to that of the thick membrane; after two hours the average total methanol produced by the thin membrane was 1.5 times higher than that produced by the thick membrane. The results demonstrate that the ability to tune the geometry of immobilized pMMO, even at the millimeter scale, impacts the performance of the biocatalytic material.

Direct Printing of PEG-pMMO Hydrogels

Projection microstereolithography (PμSL) allows 3D printing of light-curable materials by projecting a series of images on the material, followed by changing the height of the stage at discrete increments, with micron-scale resolution in all three dimensions. Therefore, it was an ideal technique for directly printing the PEG-pMMO material and determining whether changing geometrical features of the material at these length scales can influence activity. PμSL was thus used to print PEG-pMMO lattice structures with increased surface area to volume ratio due to 100 μm² vertical channels corresponding to ˜15% void volume. In this experiment, the pMMO concentration of 5 mg/ml did not attenuate the light enough for highest resolution printing; consequently, feature resolution was reduced in the z-direction and each layer of printed pMMO was exposed to multiple exposures to UV light. The pMMO activity in the printed cubic lattices with a total volume of about 27 mm³, which took approximately 50 min to print using PμSL, was reproducible but modest at 29 nmol MeOH min⁻¹ mg⁻¹. The reduction in activity compared to crude pMMO is likely due to the duration of the printing at room temperature as well as the overexposure of pMMO to UV during curing. However, the cubic lattices retained about 85% of the enzyme based on the solid volume of the lattice (23 mm³) corresponding to the highest protein loading that was have achieved. While not wishing to be bound by any theory, it is thought that this high retention was likely due to higher cross-linking efficiency.

Since the lattice geometry did not permit precise tuning of surface area to volume ratios, due to bending of lattice struts under water surface tension, a different PμSL tool designed to generate larger parts was used to print solid and hollow PEG-pMMO cylinders with surface area to volume ratios ranging from 1.47-2.33 and diameters ranging from of 1-5 mm. The hollow tube geometry may allow more facile diffusion of reactants because both the inner and outer surfaces of the cylindrical materials would be exposed. The total print time for an array of cylinders using the large-area PμSL tool was significantly reduced to ˜1 min by eliminating z-axis resolution, and the pMMO concentration was reduced to 2.3 mg/ml to allow UV light penetration through the 1.5-3 mm depth of the resin. Remarkably, the activity of pMMO in the hydrogels increased with greater surface area to volume ratios as shown in FIG. 13, with the highest ratio of 2.33 resulting in an average activity of 128+/−14 nmol MeOH min⁻¹ mg⁻¹ per cylinder, which corresponds to the highest reported physiological activity of membrane bound pMMO. The cylinders of the lowest ratio, 1.47, had an average pMMO activity of 67+/−3 nmol MeOH min⁻¹mg⁻¹. It should also be noted that the cylinders with the lowest surface area to volume ratio were only 1.5 mm in height and therefore completely submerged in the liquid phase during the activity assay, whereas all other cylinders tested were 3 mm in height and only partially submerged during the assay. Hydrogels protruding from the liquid allowed a direct interface between the gas phase and PEG-pMMO. This exposed interface likely increased the methane concentration in the PEG-pMMO material since the solubility of methane in PEG is several times higher than that in water. On average, 38% of the protein was encapsulated, although it was variable depending on the dimensions of each cylinder (27-54%). These results, combined with the results from the continuous flow reactor, indicate that an optimal pMMO material design may be hierarchical, with the smallest feature sizes at the micron scale.

Specific Methods

Materials

Reagents for buffers (PIPES, NaCl, and NaOH), HPLC grade methanol (≥99.9% purity), polyethylene glycol diacrylate 575 (PEGDA 575), and the cross-linking initiator, 2-hydroxy-2-methylpropiophenone (Irgacure® 1173), was purchased from Sigma-Aldrich (St. Louis, Mo.). All reagents were used as received. Methane gas (99.9% purity) was obtained from Matheson Tri-gas, Inc. (Basking Ridge, N.J.). pMMO concentrations were measured using the DC™ protein assay purchased from Bio-Rad (Hercules, Calif.). Lithium phenyl-2,4,6-trimethylbenzoylphosphinate (LAP) photoinitiator was synthesized following a procedure known in the art.

pMMO: Cell Growth and Membrane Isolation

Methylococcus capsulatus (Bath) cells were grown in 12-15 L fermentations. M. capsulatus (Bath) cells were grown in nitrate mineral salts medium (0.2% w/v KNO₃, 0.1% w/v MgSO₄.7H₂O and 0.001% w/v CaCl₂.2H₂O) and 3.9 mM phosphate buffer, pH 6.8, supplemented with 50 μM CuSO₄.5H₂O, 80 μM NaFe(III) EDTA, 1 μM Na₂MoO₄.2H₂O and trace metals solution. Cells were cultured with a 4:1 air/methane ratio at 45° C. and 300 rpm. Cells were harvested when the A₆₀₀ reached 5.0-8.0 by centrifugation at 5000×g for 10 min. Cells were then washed once with 25 mM PIPES, pH 6.8 before freezing in liquid nitrogen and storing at −80° C. Frozen cell pellets were thawed in 25 mM PIPES, pH 7.2, 250 mM NaCl buffer (herein referred to as pMMO buffer) and lysed by microfluidizer at a constant pressure of 180 psi. Cell debris was then removed by centrifugation at 20,000-24,000×g for one hr. The membrane fraction was pelleted by centrifugation at 125,000×g for one hour and washed 3 times with pMMO buffer before freezing in liquid nitrogen and storing at −80° C. Final protein concentrations were measured using the Bio-Rad DC™ assay. Typical storage concentrations ranged from 20-35 mg/ml.

Formation of the PEG-pMMO Hydrogels

Prior to preparation of the PEG-pMMO hydrogels, frozen as-isolated crude membranes from M. Capsulatus (Bath) (herein referred to as membrane-bound pMMO) was thawed at room temperature and used within 5 hours of thawing. Thawed membrane-bound pMMO (50-500 μg) was then mixed with PEGDA 575 in pMMO buffer at room temperature to form liquid PEG and pMMO suspensions having a final volume of 50 μl and 10-80 (v/v %) PEGDA 575. A photoinitiator (not shown in FIG. 9) was included in the suspension at 0.5 vol % with respect to PEGDA 575. The suspension was mixed by pipetting to homogeneity and then transferred to a 1 ml syringe with the tip removed. The syringe was then immediately placed under UV light at 365 nm, 2.5 mW/cm² intensity, for 3 min. After the UV exposure, the 50 μl PEG-pMMO hydrogel block was slowly pushed out of the syringe onto a tissue where it was gently blotted and then rinsed twice in pMMO buffer to remove unreacted reagents.

Activity Assay

All reactions were carried out in 2 ml glass reaction vials in pMMO buffer with 6 mM NADH as a reducing agent. Vials with 50-500 μg pMMO in 125 μl buffer solution were used as controls. For the immobilized enzyme samples, each 50 μl PEG-pMMO hydrogel block was placed in a vial and partially submerged in 75 μl buffer solution immediately after curing and rinsing. 1 ml of headspace gas was removed from each vial using a 2 ml gas tight glass syringe and replaced with 1 ml of methane, then the reaction vial was immediately placed in a heating block set at 45° C. and incubated for 4 min at 200 rpm. After 4 min, the samples were heat inactivated at 80° C. for 10 min. Samples were then cooled on ice for 20 min and pMMO control vials were centrifuged to remove the insoluble membrane fraction. For the cyclic activity assays using the PEG-pMMO immobilized enzyme, the reaction was stopped by opening and degassing the head space and immediately removing the solution for GC analysis. The block was then rinsed three times with 1 ml of pMMO buffer per wash and the assay was repeated. The amount of methanol generated during the reaction was measured by gas chromatography/mass spectrometry (GC/MS) analysis using an Agilent Pora-PLOT Q column and calibration curves were generated from methanol standards.

pMMO Flow Reactor

A simple cubic polydimethyl siloxane (PDMS) lattice with 250 micron struts and 250 micron spacing was printed using Direct Ink Write as described to provide methane permeability throughout the PEG material and to provide mechanical support. A top layer of 50 micron thick PDMS was fabricated by spin-coating Dow Corning SE-1700 PDMS diluted in toluene on a hydrophobized silicon wafer. This thin PDMS membrane prevented leakage of liquid through the membrane but provided gas permeability. Two different flow cell geometries were fabricated using polycarbonate plastic: a flow cell for a higher surface area, thin lattice (1.25 cm wide by 3 cm long) and a lower surface area, thick lattice, 1.25 by 1.25 cm. The thin lattice was 6 layers thick, and the thick lattice had 16 layers. The lattices were made hydrophilic by treating them in air plasma for 5 minutes followed by storage in deionized water. To incorporate the pMMO into the lattices, a 10 vol % concentration of PEGDA 575 was mixed with crude pMMO membrane preparations to a final concentration of 5 mg/ml pMMO. Two hundred microliters of the pMMO/PEGDA mixture were pipetted into the lattice and cured with 365 nm UV light at 2.5 mW/cm² intensity for 4 min, forming the mixed polymer (PEG/PDMS) membrane. The final concentration of pMMO in the lattices was calculated, rather than directly quantified using a protein assay, due to difficulties in quantifying the material in the lattice. The membrane was then loaded into the cell and rinsed with buffer to remove any unpolymerized material. The flow cell was placed on a hot plate calibrated with thermocouple so that the membrane would reach either 25 or 45 degrees ° C. An NADH/buffer solution (4 mg/ml NADH in PIPES pH 7.2) was prepared as the liquid phase in a 5 ml syringe, and the gas phase was prepared as 50% methane and 50% air loaded into a gas-tight 50 ml syringe. The syringes were loaded into Harvard Apparatus syringe pumps and the gas and liquid were delivered at 0.5 and 0.75 ml per hour, respectively. The gas outlet tubing was kept under 2 cm water pressure during the reaction. Fractions of liquid were collected into GC/MS autosampler vials that were kept on ice to reduce methanol evaporation and were analyzed against MeOH standards using GC/MS as described above. Methanol contamination was present in the NADH/buffer solutions, and this concentration was subtracted from the total detected in each fraction by GC/MS. No methanol contamination was found in the water used to store the PDMS. The data shown in FIG. 12B represent cumulative methanol (where the quantity of methanol produced in each fraction was added to the previous samples). Each experiment was done in triplicate; the error bars represent a standard deviation.

3D Printing of PEG-pMMO Hydrogels

The printing resin was prepared with 20 vol % PEGDA 575, 10 mg/ml LAP initiator, and 2.3-5 mg/ml crude pMMO in buffer. Using projection microstereolithography (PμSL), hydrogel blocks were printed in a cubic lattice with 100 um open channels spaced 100 μm apart and size dimensions from 1-3 mm. Solid and hollow cylinders of the same resin formulation were printed using the large area PμSL (LA PμSL) system. The cylinders had an inner diameter of 1-2.5 mm, an outer diameter of 3-5 mm, and were 1.5-3 mm high. The resin was cured with a 395 nm diode with both PμSL and LA PμS, but the intensity and exposure time varied between the systems, ranging from 11.3-20 W/cm² and 15-30 seconds per layer, respectively. Resin and printed hydrogels were stored on ice before and after the printing process. The pMMO activity assay was carried out as described above at 45° C. for 4 minutes. The methanol concentration of the activity assay and protein content of the printed hydrogels were measured as described above.

Applications/Uses

Aspects of the present invention may be used in a wide variety of applications and may provide more efficient and higher-throughput use of enzymes to catalyze chemical reactions in any potential industrial application. Illustrative applications in which aspects of the present invention may be used include, but are not limited to, fuel conversion (e.g., natural gas to liquid fuel), chemical production, pharmaceutical production, and other processes where a chemical conversion is catalyzed by enzymes, especially at phase boundaries (e.g., reaction involving a gas and a liquid, polar and non-polar species, aqueous and non-aqueous species, etc.).

The inventive concepts described herein may be used to encapsulate whole cells for biocatalysis of a range of products. In some approaches, the inventive concepts may be used with methanotrophs to upgrade methane to chemical products. In other approaches, the inventive concepts may be used with yeast to produce ethanol.

In more aspects, the inventive concepts described herein may be useful to any industry that utilizes microbes for biocatalysis, including pharmaceutical, food and beverage, chemical synthesis, waste management, and cosmetics. Inventive aspects described herein may be particularly useful for reactions that are limited by mass transfer or depend on a gas/liquid interface.

In still more aspects, the presently described inventive concepts may also be used to encapsulate engineered cell strains to produce enzymes, biological therapeutics, vaccines, and recombinant proteins that are currently produced by industrial fermentation.

In still yet more aspects, the inventive aspects described herein may be useful in applications such as tissue engineering and regenerative medicine. The invention is comprised of highly biocompatible polymers and may be printed into geometries and structures that are directly applicable to scaffolds for tissue engineering.

It should be noted that methodology presented herein for at least some of the various aspects may be implemented, in whole or in part, in computer hardware, software, by hand, using specialty equipment, etc. and combinations thereof.

Moreover, any of the structures and/or steps may be implemented using known materials and/or techniques, as would become apparent to one skilled in the art upon reading the present specification.

The inventive concepts disclosed herein have been presented by way of example to illustrate the myriad features thereof in a plurality of illustrative scenarios, aspects, and/or implementations. It should be appreciated that the concepts generally disclosed are to be considered as modular, and may be implemented in any combination, permutation, or synthesis thereof. In addition, any modification, alteration, or equivalent of the presently disclosed features, functions, and concepts that would be appreciated by a person having ordinary skill in the art upon reading the instant descriptions should also be considered within the scope of this disclosure.

While various aspects have been described above, it should be understood that they have been presented by way of example only, and not limitation. Thus, the breadth and scope of an aspect of the present invention should not be limited by any of the above-described exemplary aspects but should be defined only in accordance with the following claims and their equivalents.

What is claimed is:

1. A mixture for forming polymer-encapsulated whole cells, the mixture comprising:
a pre-polymer;
a photoinitiator; and
a plurality of whole cells.

2. The mixture as recited in claim 1, wherein the pre-polymer includes at least one pre-polymer selected from the group consisting of: poly(ethylene) glycol, amphiphilic silicones, alginate, N-isopropylacrylamide, and methacrylic acid.

3. The mixture as recited in claim 2, wherein the pre-polymer is poly(ethylene) glycol acrylate.

4. The mixture as recited in claim 2, wherein a concentration of the pre-polymer is in a range of about 10 weight % to about 50 weight % of a total weight of the mixture.

5. The mixture as recited in claim 1, wherein a molecular weight of the pre-polymer is in a range of about 575 Daltons to about 100,000 Daltons.

6. The mixture as recited in claim 1, wherein a molecular weight of the pre-polymer is in a range of about 10,000 Daltons to about 40,000 Daltons.

7. The mixture as recited in claim 1, wherein the whole cells are whole living cells.

8. The mixture as recited in claim 1, wherein the whole cells are dried whole cells.

9. The mixture as recited in claim 1, wherein the whole cells have a characteristic to convert a chemical reactant to a product, wherein the chemical reactant is a gas and the product is a liquid.

10. The mixture as recited in claim 1, wherein the whole cells are configured to convert methane to methanol.

11. The mixture as recited in claim 1, wherein the whole cells are selected from the group consisting of: methanotrophic organisms, methylotrophic organisms, and yeast.

12. The mixture as recited in claim 1, wherein a concentration of whole cells has a cell optical density in a range from about 4.0 to about 160.

13. The mixture as recited in claim 1, wherein a concentration of whole cells has a cell optical density in a range of at least 10 to about 60.

14. A product, comprising:
a structure comprising a plurality of whole cells encapsulated in a polymer, wherein the polymer is cross-linked.

15. The product of claim 14, wherein the polymer includes a poly(ethylene) glycol polymer.

16. The product of claim 14, wherein a molecular weight of the polymer is in a range of about 10,000 Daltons to about 40,000 Daltons.

17. The product of claim 14, wherein the whole cells have a characteristic to convert a chemical reactant to a product, wherein the chemical reactant is a gas and the product is a liquid.

18. The product of claim 14, wherein the whole cells are selected from the group consisting of: methanotrophic organisms, methylotrophic organisms, and yeast.

19. A bioreactor, comprising:
a three-dimensional structure, wherein the three-dimensional structure is comprised of a gas-permeable material; and
polymer-encapsulated whole cells, wherein at least one side of the three-dimensional structure is infilled with the polymer-encapsulated whole cells.

20. The bioreactor as recited in claim 19, the three-dimensional structure is a printed three-dimensional structure.

21. The bioreactor as recited in claim 20, wherein the printed three-dimensional structure is a lattice.

22. The bioreactor as recited in claim 20, wherein the printed three-dimensional structure is a tube, wherein a wall of the tube is gas-permeable, wherein an inner surface of the wall defines a center portion of the tube.

23. The bioreactor as recited in claim 22, comprising a buffer in the center portion of the tube, wherein the buffer comprises nutrients for the polymer-encapsulated whole cells.

24. The bioreactor as recited in claim 23, wherein the polymer-encapsulated whole cells comprise a plurality of living whole cells, wherein the plurality of living whole cells have a characteristic to remain viable in the bioreactor for a duration of at least five days.

25. The bioreactor as recited in claim 19, wherein a concentration of whole cells has a cell optical density in a range from about 4.0 to about 160.

26. The bioreactor as recited in claim 19, wherein a thickness of the at least one side of the three-dimensional structure is in a range of about 10 microns to about 5000 microns.

27. A method for forming the bioreactor as recited in claim 19, the method comprising:
forming the three-dimensional structure using an additive manufacturing technique;
infilling the at least one side of the three-dimensional structure with a mixture for forming the polymer-encapsulated whole cells; and
curing the three-dimensional structure infilled with the mixture.

28. The method for forming the bioreactor as recited in claim 27, wherein the three-dimensional structure is a lattice, wherein the additive manufacturing technique is selected from the group consisting of: projection microstereolithography and direct ink writing.
